(19) United States
(12) Patent Application Publication
(10) Pub. No.: US 2023/0381256 A1
(43) Pub. Date: Nov. 30, 2023

(54) AN IMMUNE CHECKPOINT-MODULATING VSV-NDV HYBRID VIRUS FOR ONCOLYTIC VIRUS IMMUNOTHERAPY OF CANCER

(71) Applicant: KLINIKUM RECHTS DER ISAR DER TECHNISCHEN UNIVERSITÄT MÜNCHEN, MUNICH (DE)

(72) Inventors: Jennifer ALTOMONTE, MUNICH (DE); Teresa KRABBE, MUNICH (DE)

(21) Appl. No.: 18/034,226

(22) PCT Filed: Oct. 29, 2021
(86) PCT No.: PCT/EP2021/080188

§ 371 Date: Apr. 27, 2023

(30) Foreign Application Priority Data
Nov. 2, 2020 (EP) 20205209.8

(51) Int. Cl. A61K 35/768; C12N 15/86; C07K 14/125; C07K 14/705; A61P 35/00

(52) U.S. Cl. CPC A61K 35/768; C12N 15/86; C07K 14/125; C07K 14/70532; A61P 35/00

(57) ABSTRACT

The present invention relates to a recombinant oncolytic virus. The present invention further relates to a nucleic acid encoding a recombinant oncolytic virus. The present invention also relates to a vector comprising a nucleic acid encoding a recombinant oncolytic virus. Furthermore, the present invention relates to a pharmaceutical composition comprising a recombinant oncolytic virus. The present invention further relates a virus, a nucleic acid, a vector, and a pharmaceutical composition for use in medicine, such as for use in the prevention and/or treatment of cancer. Moreover, the present invention relates to the use of a virus, a nucleic acid, a vector, and a pharmaceutical composition as gene delivery tool, (noninvasive) imaging of virus biodistribution, and/or for tumor detection.

### FIELD OF THE INVENTION

The present invention relates to a recombinant oncolytic virus. The present invention further relates to a nucleic acid encoding a recombinant oncolytic virus. The present invention also relates to a vector comprising a nucleic acid encoding a recombinant oncolytic virus. Furthermore, the present invention relates to a pharmaceutical composition comprising a recombinant oncolytic virus.

### BACKGROUND OF THE INVENTION

Despite many decades of intensive research, cancer remains a major worldwide health concern and imposes a heavy societal burden, both epidemiologically and financially. Cancer immunotherapy is rapidly transforming the face of medical oncology as an exciting paradigm shift in treating cancer by exploiting the immune system of the patient as the basis for the therapy. Oncolytic viruses (OVs) have recently earned their place as an exciting subset of cancer immunotherapeutics. OVs offer a novel, multi-mechanistic approach through the onset of direct tumor cell oncolysis, modulation of the tumor microenvironment, and the potential to stimulate adaptive immune responses directed against tumor cells. Therefore, OV approaches, particularly in the context of rationally designed combination regimens with other immunotherapeutics, are currently under intense investigation.

Cancer immunotherapy encompasses a wide range of approaches that have the common aim of modulating a patient's immune system to recognize and reject invading tumor cells. Vaccination approaches and adoptive immune cell therapies, such as chimeric antigen receptor (CAR) T cells, rely on the identification of suitable tumor-associated antigens (TAAs) and neoantigens to target. Due to intratumoral heterogeneity and the process of cancer immune-editing, strategies which target a single antigen can result in selection for tumor cells which do not express the targeted antigen and can escape the therapy. Furthermore, since each tumor has its own distinct gene signature, targeted therapies often require expensive and time-consuming screening of tumor biopsies and subsequent production of personalized treatments.

Immune checkpoints are compensatory controls that function to prevent constant activation of an immune inflammatory response to foreign antigens by suppressing T cell activation and creating a state of T-cell “exhaustion”. Tumor cells utilize these immune checkpoints to create an immune-suppressive microenvironment, in which they can escape immune clearance and continue to invade the host. Immune checkpoints have increasingly been considered as promising targets for cancer immunotherapy. Blockade using antibodies against key checkpoint molecules have entered the market for limited cancer indications, but only seem to be effective in small subsets of cancer patients, generally in tumors which are highly inflamed and have a high mutational load. Furthermore, the high doses of these antibodies required for systemic application can result in severe immune related toxicities.

Oncolytic viruses (OVs) offer an elegant multimodal approach to cancer therapy through their ability to cause direct tumor cell lysis, while stimulating immune responses directed against the tumor. Nevertheless, the potential of OVs as monotherapeutics is limited by the relatively rapid onset of antiviral immune responses and insufficient adoptive immune stimulation to provide systemic tumor clearance and protection from relapse. Over the last decade, significant progress has been made in the development of enhanced OV therapies, and several vectors have entered clinical trials. To date, a single OV has received Federal Drug Administration (FDA) and European Medical Association (EMA) approval for use as a clinical agent. This virus is a recombinant herpes simplex virus I (HSV-I) vector and is currently approved only for one single indication which is non-resectable melanoma. Furthermore, U.S. Ser. No. 10/604,574 B2 relates to oncolytic viral delivery of therapeutic polypeptides. However, clinical trial results are often disappointing due to inadequate tumor responses to most OV therapies in immune competent hosts.

The present inventors have previously engineered a hybrid oncolytic virus technology (WO 2017/198779), which combines the beneficial features of oncolytic vesicular stomatitis virus (VSV) with those of Newcastle disease virus (NDV), while eliminating the safety concerns of each.

Recently, a strategy to combine OVs with immune checkpoint therapies has emerged. The rationale is that the local virus infection within the tumor can transform the “cold” tumor microenvironment to an inflamed, “hot” environment marked by high levels of cytokines and immune cell infiltration, thereby sensitizing the tumor to the effects of immune checkpoint blockade. It has been postulated that tumor therapy with oncolytic viruses leads to upregulation of the ligand, PD-L1, of the co-inhibitory T-cell receptor, programmed cell death protein 1 (PD-1), within the tumor, and that blockade of the interaction between PD-1 and PD-L1 enhances the oncolytic immunotherapeutic effect. A strategy utilizing an oncolytic myxoma virus expressing sPD-1 has been published [1].

However, various hurdles must be addressed in order to fully exploit the potential of the combination of OV with an immune checkpoint therapy. Firstly, a challenge is that conventional immune checkpoint modulating drugs consist of antibodies, which need to be administered systemically and at high concentrations, often leading to intolerable side effects. Secondly, the prohibitively high costs of immunotherapeutics substantially limit the feasibility of combining two individual drug products in this class. Thirdly, in order to optimize the synergistic effects, a challenge is to choose an efficient oncolytic virus, which combines a potent tumor debulking effect with immunogenic cell death and inflammation.

Thus, there is a need in the art for improved means and methods for oncolytic virotherapy as well as for improved oncolytic viruses. The aim of the present invention is to provide a superior cancer therapeutic that combines optimal viral-mediated oncolysis with potent immune-mediated effects, in the absence of systemic toxicity.

### SUMMARY OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the present invention relates to a recombinant oncolytic virus,
comprising a vesicular stomatitis virus (VSV),
wherein the glycoprotein (G protein) of VSV is deleted, and which comprises
   a modified fusion protein (F protein) of Newcastle disease virus (NDV), and
   the hemagglutinin neuraminidase (HN) protein of NDV, further comprising soluble PD-1 (sPD-1).

In one embodiment, said recombinant virus further comprises a Fe domain or a fragment thereof, preferably a Fe domain of human IgG1, IgG2, IgG3, and/or IgG4, or a fragment thereof.

In one embodiment, said Fe domain or fragment thereof is fused to said sPD-1.

In one embodiment, said sPD-1 is a high affinity sPD-1 (HA-sPD-1), preferably having an affinity to its ligands PD-L1 and PD-L2 which is at least 2-fold higher, such as 45- and 30-fold higher, respectively, than an affinity of a wildtype sPD-1 to said ligands, and/or has an affinity with a K_{d} of <3.2 μM with regard to PD-L1 and/or <0.1 μM with regard to PD-L2.

In one embodiment, said sPD-1 comprises a mutation at a position selected from 132 and 41 of SEQ ID NO. 2, preferably a mutation selected from A132L, L41I, and L41V.

In one embodiment, said sPD-1 comprises or consists of a sequence having SEQ ID NO. 2, and optionally further comprises a mutation at a position selected from 132 and 41 of SEQ ID NO. 2, preferably a mutation selected from A132L, L41I, and L41V.

In one embodiment, said sPD-1 is HA-sPD-1-A132L having a sequence of SEQ ID NO. 3.

In one embodiment, the modified fusion protein (F protein) of NDV is the F3aa-modified F protein having a sequence of SEQ ID NO. 25,

and/or comprises at least one amino acid substitution in the protease cleavage site, preferably in position L289 of SEQ ID NO. 25, more preferably L289A;

and/or the modified fusion protein (F protein) of NDV is the F3aa-modified F protein with an amino acid substitution L289A having SEQ ID NO. 4;

and/or the G protein of VSV is replaced by the modified fusion protein having a sequence of SEQ ID NO. 4 and HN protein of NDV having a sequence of SEQ ID NO. 5.

In a further aspect, the present invention relates to a nucleic acid encoding a recombinant oncolytic virus as defined above.

In one embodiment, said nucleic acid comprises a nucleic acid encoding a Fe domain or fragment thereof, preferably having a sequence of SEQ ID NO. 7, which is fused to a nucleic acid encoding said sPD-1, preferably having a sequence of SEQ ID NO. 6, wherein, preferably, a fusion product thereof has a nucleic acid sequence of SEQ ID NO. 8.

In this aspect, said recombinant oncolytic virus, said Fe domain, said fragment, and said sPD-1 are as defined above.

In a further aspect, the present invention relates to a vector comprising a nucleic acid as defined above, preferably having a sequence of SEQ ID NO. 9, optionally further comprising any of
a reporter gene,
   such as any of HSV1-sr39TK, sodium iodide symporter (NIS), somatostatin receptor 2 (SSTR2), luciferase (Firefly or Renilla), green fluorescence protein (GFP), lacZ, and tyrosinase;
a gene to be delivered to a tumor cell and/or tumor tissue, such as any of
   an immune stimulating gene, e.g. IFN-α, IFN-β, or granulocyte macrophage colony-stimulating factor (GM-CSF), IL-12, or IL-15;
   an immune checkpoint inhibitory antibody, e.g. PD-1, PD-1-L, CTLA-4, LAG-3, or B7-H3; and/or
   a tumor associated antigen (TAA) for vaccination (specific for the tumor being targeted);
or combinations thereof.

In a further aspect, the present invention relates to a nucleic acid as defined above or a vector as defined above, comprising or consisting of the nucleotide sequence of SEQ ID NO. 9 or 15,
or a nucleotide sequence having at least 60%, preferably at least 70% or 80%, more preferably at least 90% or 95% sequence identity to the nucleotide sequence of SEQ ID NO. 9 or 15,

or comprising or consisting of the nucleotide of SEQ ID NO. 16,
or a nucleotide sequence having at least 60%, preferably at least 70% or 80%, more preferably at least 90% or 95% sequence identity to the nucleotide sequence of SEQ ID NO. 16.

In a preferred embodiment, the nucleic acid, as defined above, or the vector, as defined above, comprise or consist of the nucleotide sequence of SEQ ID NO. 9 or 15, or a nucleotide sequence having at least 60%, preferably at least 70% or 80%, more preferably at least 90% or 95% sequence identity to the nucleotide sequence of SEQ ID NO. 9 or 15.

In a further aspect, the present invention relates to a pharmaceutical composition, comprising
(i) the recombinant oncolytic virus as defined above, the nucleic acid as defined above, or the vector as defined above: and
(ii) optionally, pharmaceutically acceptable carrier(s) and/or excipient(s),
(iii) optionally, a further drug, such as
   a chemotherapeutic agent,
   a radiotherapeutic agent,
   a tumor vaccine,
   an immune checkpoint inhibitor, such as anti-CTLA4,
   an adoptive cell therapy system, such as T cells or dendritic cells,
   a cell carrier system,
   a small molecule inhibitor,
   an embolization agent,
   a shielding polymer.

In one embodiment, the pharmaceutical composition as defined above is formulated for any of systemic delivery, tumor injection, intravenous administration, and intra-arterial administration,

and/or for an intradermal, subcutaneous, intramuscular, intravenous, intraosseous, intraperitoneal, intrathecal, epidural, intracardiac, intraarticular, intracavernous, intracerebral, intracerebroventricular, or intravitreal injection.

In a further aspect, the present invention relates to a recombinant oncolytic virus, as defined above, a nucleic acid, as defined above, a vector, as defined above, or a pharmaceutical composition, as defined above, for use in medicine.

In a further aspect, the present invention relates to a recombinant oncolytic virus, as defined above, a nucleic acid, as defined above, a vector, as defined above, or a pharmaceutical composition, as defined above, for use in the prevention and/or treatment of cancer, such as in the prevention and/or treatment of a cancer selected from hepatocellular carcinoma, pancreatic cancer, and melanoma.

In one embodiment, the recombinant oncolytic virus for use, as defined above, the nucleic acid for use, as defined above, the vector for use, as defined above, or the pharmaceutical composition for use, as defined above, is used in combination with other therapies, such as
cell carrier systems, e.g. T cells, dendritic cells, NK cells, mesenchymal stem cells,
immunotherapies, e.g. tumor vaccines or immune checkpoint inhibitors,
adoptive cell therapies, such as with T cells or dendritic cells, and/or
standard tumor therapies, e.g. radiofrequency ablation, chemotherapy, embolization, small molecule inhibitors.

In a further aspect, the present invention relates to a recombinant oncolytic virus, as defined above, a nucleic acid, as defined above, a vector, as defined above, or a pharmaceutical composition, as defined above, for use in the diagnosis of cancer, such as in the diagnosis of a cancer selected from hepatocellular carcinoma, pancreatic cancer, and melanoma.

In a further aspect, the present invention relates to a use of the recombinant oncolytic virus, as defined above, or the nucleic acid, as defined above, or the vector, as defined above, or the pharmaceutical composition, as defined above, as gene delivery tool, (noninvasive) imaging of virus biodistribution, and/or for tumor detection. In one embodiment, such use is an in-vitro-use.

In a further aspect, the present invention relates to a method of diagnosis, prevention and/or treatment of a cancer comprising administering to a subject in need thereof a therapeutically amount of the recombinant oncolytic virus, as defined above, or the nucleic acid, as defined above, or the vector, as defined above, or the pharmaceutical composition, as defined above.

In one embodiment, said administering is systemic, intravenous, intra-arterial, via injection into tumor, and/or via intradermal, subcutaneous, intramuscular, intravenous, intraosseous, intraperitoneal, intrathecal, epidural, intracardiac, intraarticular, intracavernous, intracerebral, intracerebroventricular and intravitreal injection(s).

In this aspect, said cancer and said administering are as defined above.

In a further aspect, the present invention relates to a use of a recombinant oncolytic virus, as defined above, or a nucleic acid, as defined above, or a vector, as defined above, or a pharmaceutical composition, as defined above, for the manufacture of a medicament for diagnosing, preventing and/or treating cancer. In this aspect, said cancer and said diagnosing, preventing and/or treating are as defined above.

### DETAILED DESCRIPTION

The enhanced virus of the present invention, which is a modified VSV-NDV, offers several beneficial features over conventional oncolytic viruses, such as rapid and efficient tumor cell oncolysis generated by the induction of cell-cell fusion reactions, while maintaining an exceptional safety profile. Not only do the infected cells fuse with neighboring tumor cells, thereby killing them, but they also set off a series of events which causes the patient's immune system to launch an attack on remaining uninfected tumor cells, creating an inflammatory tumor microenvironment. Since VSV-NDV can replicate well in all tumor cells, it has far-reaching therapeutic effects in a multitude of tumor indications.

The present invention provides a potent oncolytic virus with an effective immune checkpoint blocking molecule within a single therapeutic agent. The present inventors use the safe and immune-stimulating rVSV-NDV virus as a vector to express a soluble PD-1 (sPD-1) molecule, containing the signaling domain and the soluble extracellular domain of human PD-1. The recombinant vector infects tumor cells, where it replicates and evokes secretion of sPD-1.

Then, sPD-1 attaches to its ligands, PD-L1 and PD-L2, on the surface of surrounding tumor cells and immune cells (i.e. dendritic cells), and thereby acts as a decoy to compete for T cell binding to their inhibitory ligands, thereby allowing the T cells to remain active, without the need for antibodies (FIG. 1).

Compared to traditional immune checkpoint blockade (ICB) therapy via antibodies, the invention is inventive in that it combines oncolytic virus therapy and ICB into a single therapeutic agent, which acts to simultaneously debulk the tumor, via direct oncolytic effects, while alleviating the immune suppression within the microenvironment. Furthermore, this approach allows self-amplification of therapeutic agents through virus replication and local release of the PD-1 directly at the tumor site, which is a major safety benefit compared to an antibody approach.

The present invention has the advantages that, firstly, the vector contains a highly fusogenic NDV fusion (F) protein, such as NDV/F3aa(L289A), which induces a potent immunogenic cell death and optimally synergizes with immune checkpoint blockade; secondly, the sPD-1 used in the construct is preferably a “high affinity” version having increased affinity compared to wild type sPD-1. Such high affinity version is, for example, produced via an introduction of a single alanine to leucine substitution at amino acid 132 (A132L) to result in a 45- and 30-fold higher affinity binding to its ligands, PD-L1 and PD-L2 [2], respectively, or is, for example, a high affinity consensus variant encoding an isoleucine or valine at position 41 having a 40,000-fold higher affinity for PD-L1 than the wildtype [3]. Thirdly, HA-sPD-1 is fused to the Fe domain of human IgG for enhanced stability. In one embodiment, the Fc region directly follows the sPD-1 sequence in frame, wherein the stop codon of sPD-1 is removed in order to generate a sPD-1-Fc fusion protein with a Fe domain. In one embodiment, a high affinity sPD-1 (HA-sPD-1) has an increased affinity compared to wild type sPD-1 and/or compared to wild type PD-1, such as a twofold increase in the affinity to the respective ligand(s). In one embodiment, a “high affinity” version of sPD-1 has an affinity which is at least twice as high as the affinity of wild type sPD-1, such as an 5 times higher affinity, 10 times higher affinity, 30 times higher affinity, or 45 times higher affinity. In one embodiment, a wild type human PD-1 has a K_{d} of 6.36 μM and 0.19 μM with regard to PD-L1 and PD-L2, respectively. In one embodiment, a HA-sPD-1 (high affinity sPD-1) has an affinity with a K_{d} of <3.2 μM with regard to PD-L1 and <0.1 μM with regard to PD-L2, preferably an affinity with a K_{d} of <0.5 μM with regard to PD-L1 and <0.01 μM with regard to PD-L2, such as 0.14 μM and 0.0065 μM with regard to PD-L1 and PD-L2, respectively.

The binding of the wildtype sPD-1 to its ligand is likely too weak to support its application as a therapeutically effective approach. The advantage of the present invention is that the high affinity version of sPD-1 used in a vector of the present invention is more effective and thus has higher efficiency in cancer treatment.

The vector of the present invention offers several advantages over antibody-based ICB therapies. For example, it offers a local release of PD-1 specifically at the tumor site, which has the potential to avoid the severe side effects that are attributed to the systemic delivery of these antibodies. It also provides a synergistic mechanism of action through tumor debulking, induction of antitumor immune responses, and therapeutic modulation of immune suppression within the tumor microenvironment. Furthermore, the rVSV-NDV-sPD-1 drug product can potentially be produced for a fraction of the price of PD-1 antibodies, and it is more cost-effective than administration of combination therapy of an OV in addition to a separate PD-1 antibody. Furthermore, the vector of the present invention is advantageously safe for human use.

The present invention has further advantages, such as advantages over an oncolytic myxoma virus expressing a sPD-1. Firstly, the rVSV-NDV vector is an optimal oncolytic virus platform, as it is extremely safe and highly effective in tumor killing through direct oncolytic effects and induction of highly immunogenic cell death, leading to abscopal effects. These superior therapeutic effects are attributed to the VSV glycoprotein substitution with the envelope proteins of NDV (comprising the fusion (F) and hemagglutinin neuraminidase (HN) proteins), and more specifically, through the use of the modified hyperfusogenic fusion (F) protein that is engineered into the virus construct. In one embodiment, the modified fusion protein (F protein) of NDV is the F3aa-modified F protein e.g. having a sequence of SEQ ID NO. 25, optionally further comprising at least one amino acid substitution in the protease cleavage site, preferably in position L289 of SEQ ID NO. 25, more preferably L289A. In one embodiment, using the L289A-modified version of the NDV fusion (F) protein, namely NDV/F3aa(L289A), such as a protein having an amino acid sequence of SEQ ID NO. 4, provides increased hyperfusogenicity. These properties are the basis for the rVSV-NDV being an ideal OV vector for combination with ICB. Moreover, the rVSV-NDV-sPD-1 vector utilizes sPD-1, preferably a high affinity version of human sPD-1, which offers a stronger interaction of the sPD-1 with its ligands and thus a greater potential for therapeutic effects. Finally, a high affinity sPD-1 is fused with a Fe domain of human IgG, which allows for greater stability for an improved pharmacokinetic profile in vivo. In one embodiment, a nucleic acid and/or a vector of the invention comprise a nucleotide sequence having SEQ ID NO. 9 or 15, wherein SEQ ID NO. 9 and 15 both encode VSV-NDV-HAsPD1-Fe, wherein SEQ ID NO. 9 further comprises non-coding regions.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “20 to 100 nucleotides” should be interpreted to include not only the explicitly recited values of 20 to 100, but also include individual values and sub-ranges within the indicated range. Thus, included in this numerical range are individual values such as 20, 21, 22, 23, 24, 25, 26, 27, 28, 29, . . . 97, 98, 100 and sub-ranges such as from 25 to 35, from 20 to 40, from 25 to 50, etc. This same principle applies to ranges reciting only one numerical value, such as “at least 25 nucleotides”. Furthermore, such an interpretation should apply regardless of the breadth of the range or the characteristics being described.

The term “VSV”, as used herein, relates to Vesicular stomatitis virus (VSV) which is a negative-strand RNA virus of the Rhabdovirus family. VSV vectors are very attractive oncolytic agents due to their inherent tumor specificity and rapid replication cycle, which results in high intratumoral titers and subsequent tumor cell lysis. The genome of VSV is a single molecule of negative-sense RNA that encodes five major proteins: glycoprotein (G), large polymerase protein (L), phosphoprotein (P), matrix protein (M) and nucleoprotein (N). The total genome is about 11,000 nucleotides. The VSV G protein enables viral entry. It mediates viral attachment to an LDL receptor (LDLR) or an LDLR family member present on the host cell. Following binding the VSV-LDLR complex is rapidly endocytosed. It then mediates fusion of the viral envelope with the endosomal membrane. VSV enters the cell through partially clathrin-coated vesicles; virus-containing vesicles contain more clathrin and clathrin adaptor than conventional vesicles. Virus-containing vesicles recruit components of the actin machinery for their interaction, thus inducing its own uptake. Replication occurs in the cytoplasm. The VSV L protein is encoded by half the genome, and combines with the phosphoprotein to catalyze replication of the mRNA. The VSV M protein is encoded by an mRNA that is 831 nucleotides long and translates to a 229 amino acid-protein. The predicted M protein sequence does not contain any long hydrophobic or nonpolar domains that might promote membrane association. The protein is rich in basic amino acids and contains a highly basic amino terminal domain. The term “rVSV” relates to recombinant vesicular stomatitis virus (VSV).

VSV Indiana complete genome SEQ ID NO. 17

NCBI GenBank accession No. J02428.1

VSV Indiana G protein SEQ ID NO. 18

See GenBank accession No. X03633.1 for nucleotide and amino acid sequence.

The term “NDV”, as used herein, relates to Newcastle disease virus (NDV) which is an avian virus of the Paramyxovirus family. Members of this family have a single stranded linear RNA. The total genome is about 16,000 nucleotides. Replication of the virus takes place in the cytoplasm of the host cell. It is a negative-stand RNA virus and has been developed as an oncolytic virus, due to its innate ability to replicate and cause lysis in tumor cells, while leaving healthy cells unharmed. Phase I-II clinical trials suggest that there is minimal toxicity related to the therapy. A major benefit of NDV as an oncolytic agent is that the viral envelope, which is comprised of a hemagglutinin-neuraminidase (HN) and fusion (F) protein, mediates not only virus attachment and fusion to the target cell, but it causes fusion of infected cells to their neighboring uninfected cells, providing a potent mechanism for viral spread and tumor cell killing. Furthermore, new evidence indicates that the syncytia formation caused by cell-cell fusion results in a multimodal cell death response, which can synergize with the direct oncolytic effect of the virus for a potent mechanism of tumor destruction.

Two proteins of Newcastle disease virus are inserted in the envelope. They are the haemagglutinin/neuraminidase protein (HN) and the fusion protein (F). These two proteins are important in determining the virulence of the virus and how the virus infects host cells. The haemagglutinin/neuraminidase protein has two sections that are of interest: (1) The haemagglutinin section, which is an attachment protein and binds to receptors on the outside of the membrane of host cells including red blood cells. (2) The neuraminidase section is the active site of an enzyme that aids in the release of the virus from the membrane of host cells. The activity of this enzyme affects the time taken for the virus to elute from red blood cells.

The fusion protein F fuses the virus envelope to the membrane of the host cell. This allows penetration of the host cell by the viral genome. In order for fusion to occur, the shape of the native fusion protein must be changed. This change happens when a host cell protease cleaves the protein at a specific cleavage site. After this has happened, the fusion protein is activated and can then fuse to the membrane of the cell. The sequence of the amino acids around the cleavage site determines the range of proteases that can activate cleavage of the protein. This sequence therefore determines the virulence. NDV F protein is responsible for viral fusion with the cell membrane and for viral spread from cell to cell via formation of syncytia. The presence of a multibasic cleavage site within the F protein allows for protein cleavage and activation by a broad range of proteases and is a determinant of virulence in velogenic viral strains. The inventors have previously demonstrated that a single amino acid substitution from leucine to alanine at amino acid 289 (L289A) in the F3aa-modified fusion protein results in substantially greater syncytial formation and tumor necrosis than the virus bearing only the F3aa mutation, without any additional toxicity [4]. The fusogenic and oncolytic activity of the rNDV/F3aa strain can be further enhanced by a point mutation in the F protein at residue 289 from leucine to alanine, generating rNDV/F3aa (L289A). In an orthotopic immunocompetent liver tumor rat model, administration of the mutant virus via hepatic arterial infusion resulted in significant syncytia formation and necrosis, which translated to a significant 20% prolongation of survival over treatment with the original rNDV/F3aa virus [4].

NDV Hitchner B1 complete genome SEQ ID NO. 20

GenBank accession No. AF375823

NDV HN protein SEQ ID NOs. 5 and 19

See GenBank accession No. AF375823 and NCBI Gene ID 912270 for nucleic acid and amino

acid sequence.

NDV F protein SEQ ID NOs. 21 and 22

See GenBank accession No. AF375823 and NCBI Gene ID 912271 for nucleic acid and amino acid sequence.

NDV F3aa-modified fusion protein SEQ ID NOs. 24 and 25

NDV F3aa-modified fusion protein with L289A SEQ ID NOs. 23 and 4

As discussed above, the present invention provides recombinant oncolytic VSV viruses, wherein the glycoprotein protein of VSV is pseudotyped, and which further comprise a soluble PD-1. In one embodiment, when referring to a virus “further comprising soluble PD-1”, this is preferably meant to refer to a scenario wherein a nucleic acid of said virus, preferably a genome of said virus, encodes said soluble PD-1; e.g. a viral genome of VSV and/or VSV-NDV encodes soluble PD-1. For example, a nucleic acid encoding said soluble PD-1 can be incorporated into a viral genome, e.g. a genome of VSV or a VSV-NDV hybrid virus, by genetic modification. In one embodiment, from such viral genome said soluble PD-1 is expressed, e.g. by means of a cell such as a cancer cell. In one embodiment, the recombinant oncolytic virus further comprising soluble PD-1 comprises a nucleic acid sequence encoding said soluble PD-1. In one embodiment, the recombinant oncolytic virus further comprises soluble PD-1 as a part of the viral genome of the recombinant oncolytic virus, preferably as a part of the viral genome of VSV. In one embodiment, soluble PD-1 is expressed as a recombinant protein. In one embodiment, a cell such as a cancer cell infected with said recombinant oncolytic virus expresses said soluble PD-1. In one embodiment, the recombinant oncolytic virus further comprising soluble PD-1 is a recombinant oncolytic virus encoding said soluble PD-1; preferably encoding said soluble PD-1 in a genome of said virus. The concept of exchanging the glycoprotein (“pseudotyping”) of a virus with that of a heterologous virus has previously been demonstrated as an effective means of altering virus tropism. Using this approach, the viral backbone is kept intact, and therefore, virus replication in susceptible cells should be minimally effected.

In one embodiment, the G protein of VSV is replaced by the modified fusion protein, preferably the modified fusion (F) protein having an amino acid substitution at position L289 e.g. L289A, and HN protein of NDV. In one embodiment, the recombinant oncolytic virus furthermore comprises the remaining proteins of VSV, namely the large polymerase protein (L), phosphoprotein (P), matrix protein (M) and nucleoprotein (N). For example, the endogenous glycoprotein of VSV can be deleted from a plasmid encoding the full-length VSV genome. The NDV glycoprotein, comprising a modified fusion protein (NDV/F(L289A)) and hemagglutinin-neuraminidase (NDV/HN), can be inserted as discrete transcription units between the VSV matrix (M) and large polymerase (L) genes (see FIG. 2A and WO 2017/198779). In one embodiment, a modified fusion protein (F protein) of NDV is a F3aa-modified F protein, optionally comprising at least one amino acid substitution in the protease cleavage site, preferably in position L289, such as L289A.

In an embodiment of the vector, preferably recombinant VSV (rVSV) vector, of the present invention, the modified fusion protein (F protein) of NDV comprises or consists of the amino acid sequence of SEQ ID NO. 25 [=aa sequence of F3aa protein] or SEQ ID NO. 4 [=aa sequence of F3aa protein/L289A], or an amino acid sequence having at least 60%, or preferably at least 70% or 80% or 90% or 95% sequence identity to the amino acid sequence of SEQ ID NOs. 25 or 4, and/or wherein the modified fusion protein (F protein) of NDV is encoded by a nucleotide sequence of SEQ ID NO. 24 [=nucleotide sequence of F3aa protein] or SEQ ID NO. 23 [=nucleotide sequence of F3aa protein/L289A], or a nucleotide sequence having at least 60%, or preferably at least 70% or 80% or 90% or 95% sequence identity to the nucleotide sequence of SEQ ID NOs. 24 or 23.

In an embodiment of the vector, preferably rVSV vector, of the present invention, the HN protein of NDV comprises or consists of the amino acid sequence of SEQ ID NO. 5, or an amino acid sequence having at least 60%, or preferably at least 70% or 80% or 90% or 95% sequence identity to the amino acid sequence of SEQ ID NO. 5, and/or the HN protein of NDV is encoded by a nucleotide sequence of SEQ ID NO. 19 or a nucleotide sequence having at least 60%, or preferably at least 70% or 80% or 90% or 95% sequence identity to the nucleotide sequence of SEQ ID NO. 19.

The present invention comprises nucleic acids encoding the oncolytic viruses of the present invention. The present invention further comprises vectors comprising the nucleic acids of the present invention.

In one embodiment, the vector of the present invention further comprises any of
a reporter gene,
   such as any of HSV1-sr39TK, the sodium iodide symporter (NIS), somatostatin receptor 2 (SSTR2), luciferase (Firefly or Renilla), green fluorescence protein (GFP), lacZ, and tyrosinase,
a gene to be delivered to a tumor cell and/or tumor tissue, such as any of
   an immune stimulating gene, e.g. IFN-α, IFN-β, or granulocyte macrophage colony-stimulating factor (GM-CSF), IL-12, or IL-15;
an immune checkpoint inhibitory antibody, such as PD-1, PD-L1, CTLA-4,
   LAG-3, or B7-H3; and
   a tumor associated antigen (TAA) for vaccination (specific for the tumor being targeted);
and combinations thereof.

The present invention provides the recombinant oncolytic viruses, the nucleic acids of the present invention, the vectors of the present invention, and/or the pharmaceutical composition of the present invention for use in medicine. The present invention further provides the recombinant oncolytic viruses, the nucleic acids of the present invention, the vectors of the present invention, and/or the pharmaceutical composition of the present invention for use in the diagnosis, prevention and/or treatment of cancer. The present invention also provides the recombinant oncolytic viruses, the nucleic acids of the present invention, the vectors of the present invention, and/or the pharmaceutical composition of the present invention for use in oncolytic therapy, particularly oncolytic virotherapy. The term “oncolytic virotherapy”, as used herein, refers to therapy of cancer by administration of oncolytic viruses, nucleic acids encoding them or respective vectors to induce tumor regression. In one embodiment, the recombinant oncolytic viruses of the present invention, the nucleic acids of the present invention, the vectors of the present invention, and/or the pharmaceutical composition of the present invention are provided for use in combination with other therapies, such as
cell carrier systems, e.g. T cells, dendritic cells, NK cells, mesenchymal stem cells, immunotherapies, e.g. tumor vaccines or immune checkpoint inhibitors, adoptive cell therapies, e.g. with T cells or dendritic cells, and/or standard tumor therapies, e.g. radiofrequency ablation, chemotherapy, embolization, small molecule inhibitors.

In one embodiment, an administration of a recombinant oncolytic virus, a nucleic acid, a vector, and/or a pharmaceutical composition to a patient in need thereof is systemic, intravenous, intra-arterial, via injection into tumor, and/or via intradermal, subcutaneous, intramuscular, intravenous, intraosseous, intraperitoneal, intrathecal, epidural, intracardiac, intraarticular, intracavernous, intracerebral, intracerebroventricular, and intravitreal injection(s). In a preferred embodiment, the pharmaceutical composition comprising the recombinant oncolytic virus of the present invention is formulated as a liquid composition for administration, for example intravenous or intratumoral administration. Such composition is administered to a patient in need thereof at a suitable interval, such as once in a week for a period of 1 to 15 weeks.

The present invention further provides a method of diagnosis, prevention and/or treatment of cancer comprising the step of administering to a subject in need thereof a therapeutically effective amount of the recombinant oncolytic virus, the nucleic acid or the vector of the present invention or the pharmaceutical composition of the present invention. A therapeutically effective amount of a recombinant oncolytic virus, nucleic acid or vector of the present invention is the amount which results in the desired therapeutic result, in particular tumor regression.

The recombinant viruses, nucleic acids, vectors or their pharmaceutical composition(s) are preferably administered in multiple cycles over a period of time, such as for several days up to several weeks. In one embodiment, a pharmaceutical composition of the present invention comprises carrier(s) and/or excipient(s), such as a physiological buffer, and/or a polymer and/or lipid for improved stability and tumor transduction efficiency.

The present invention also relates to an in vitro and/or ex vivo use of the recombinant oncolytic virus, nucleic acid, vector, and/or pharmaceutical composition of the invention as gene delivery tool, (noninvasive) imaging of virus biodistribution, and/or for tumor detection.

The vector of the present invention comprises, inserted into the VSV G-deleted vector, a modified hyperfusogenic F protein together with a NDV HN attachment protein. By creating a hybrid of these two potent oncolytic vectors, the positive features of each virus were merged, while simultaneously eliminating the safety concerns of each. The resulting vector has the VSV backbone and, therefore, maintains the rapid replication cycle of wildtype VSV. Furthermore, due to the incorporation of the HN and hyperfusogenic F proteins of NDV, the recombinant virus induces enhanced syncytia formation, allowing for efficient intratumoral spread of the virus and a potent mechanism of tumor cell death and induction of antitumor immune responses. Using this strategy, the benefit of a fusogenic virus can be achieved without the environmental threat associated with NDV. Furthermore, since the endogenous VSV glycoprotein has been deleted, there is no neurotoxicity associated with the vector. Finally, since NDV attaches to target cells via sialic acid residues, which are upregulated on tumor cells, the present invention achieves additional transductional tumor targeting with the pseudotyped vector. The specific virus modification allows to provide a highly potent virus due to the substitution of the VSV envelope protein with that of NDV, in addition to the virus being safer. A mutated version of the NDV F protein has been introduced for further improving the efficacy of the resulting recombinant virus, without negatively impacting safety. In additional thereto, the vector further comprises the highly affine soluble PD-1 which allows for a highly efficient inhibition of an immune checkpoint.

The benefit of the vector having a glycoprotein exchange is three-fold:
1. The neurotropism associated with the endogenous VSV glycoprotein can be averted by the deletion of the VSV envelope and the introduction of the non-neurotropic NDV envelope proteins;
2. Tumor cells can be targeted via upregulation of sialic acid residues, which are the natural receptor for NDV; and
3. Viral spread and tumor cell killing can be significantly enhanced via introduction of the highly fusogenic mutant version of the NDV F protein.

The virus of the present invention offers improved safety and enhanced efficacy over other vectors. Furthermore, using the rVSV-NDV vector backbone is advantageous due to its hyperfusogenic feature, lack of pre-existing immunity in the general population, and no expected attenuation compared to VSV or NDV. Furthermore, an advantage of fusion of sPD-1 with Fe is, firstly, that the stability of sPD-1 is increased and, secondly, that the Fe domain enables the complex to interact with Fc receptors on immune cells, which further contributes to the immune-therapy. Preferably, the recombinant oncolytic virus of the present invention is a recombinant oncolytic vesicular stomatitis virus (VSV). In one embodiment, the recombinant oncolytic virus of the present invention comprises an oncolytic virus, as defined in WO 2017/198779, further comprising soluble PD-1 (sPD-1), preferably high affinity sPD-1 (HA-sPD-1), such as a high affinity sPD-1 variant having SEQ ID NO. 3, optionally further comprising a Fe domain, wherein said optional Fe domain is preferably fused to said sPD-1. In one embodiment, the recombinant oncolytic virus of the present invention comprises an oncolytic virus, as defined in WO 2017/198779, further comprising sPD-1, preferably a high affinity sPD-1 (HA-sPD-1), such as a high affinity sPD-1 variant having SEQ ID NO. 3, further comprising a Fe domain which is fused to said sPD-1. In one embodiment, the virus of the present invention comprises a N protein, P protein, M protein, and L protein of VSV, a modified F protein and HN protein of NDV, and a HA-sPD-1-Fc fusion protein and/or genes encoding such proteins.

The term “Fe domain”, as used herein, relates to the fragment crystallizable region of an antibody which is the tail region of an antibody that interacts with cell surface receptors, such as Fc receptors and some proteins of the complement system. In one embodiment, a Fe domain is any of a human IgG1-Fc domain, IgG2-Fc domain, IgG3-Fc domain, and IgG4-Fc domain. In one embodiment, when referring to a “Fe domain” and/or to a “Fe domain or fragment thereof”, the term also comprises biologically functional fragments of a Fe domain, such as a CH3 fragment of a FC domain, i.e. the term comprises entire Fe domains and fragments thereof, such as CH3 fragments. A fragment of a Fe domain is typically a biologically functional fragment of a Fe domain, i.e. the fragment has the capacity of interacting with cell surface receptors, such as Fc receptors, and/or of interacting with components of the complement system, for example a CH3 fragment.

In one embodiment, a patient's response to infection with the recombinant virus of the present invention induces a highly inflamed tumor microenvironment, thereby sensitizing the tumor to immune checkpoint inhibition. In one embodiment, the terms “subject” and “patient” are used interchangeably, and preferably relate to a mammalian patient, more preferably a human patient.

In one embodiment, the term “soluble PD-1” relates to a soluble form of programmed cell death protein 1 (PD-1). PD-1 is an immune checkpoint, namely a protein that has a role in regulating the immune system's response by down-regulating the immune system and promoting self-tolerance by suppressing T cell inflammatory activity. PD-1 has two ligands, namely PD-L1 and PD-L2, which are members of the B7 family. PD-1 typically has a sequence as shown in SEQ ID NO. 1. PD-1 is a protein on the surface of cells and soluble PD-1 is a soluble form thereof which can be secreted from cells. Soluble PD-1 typically has a sequence as shown in SEQ ID NO. 2. In one embodiment, soluble PD-1 is a high affinity sPD-1 having a sequence of SEQ ID NO. 3. In one embodiment, a virus of the present invention and/or a pharmaceutical composition of the present invention comprise(s) sPD-1 having at least 60%, preferably 80%, more preferably 95% or more such as 99%, sequence identity to an amino acid sequence having any of SEQ ID NO. 1-3, preferably having SEQ ID NO. 3. In one embodiment, a virus of the present invention, a nucleic acid of the present invention, a vector of the present invention, and/or a pharmaceutical composition of the present invention comprise(s) sPD-1 having at least 60%, preferably 80%, more preferably 95% or more such as 99%, sequence identity to a nucleic acid encoding an amino acid sequence having any of SEQ ID NO. 1-3, preferably having SEQ ID NO. 3, such as a nucleic acid having a sequence of SEQ ID NO. 6. Due to the codon redundancy of the genetic code, when referring to a nucleic acid, alternative nucleic acid sequences are also encompassed which encode the same amino acid sequence but which comprise alternative codons.

The term “fused” and/or “fusion protein”, as used herein in the context of sPD-1 and Fe, relates to the fusion of proteins, such as sPD-1 and Fe, created through the joining of two or more genes that originally coded for separate proteins. Translation of such a fusion gene, for example sPD-1-Fe, results in a single or multiple polypeptides with functional properties derived from each of the original proteins. Fusion of proteins typically involves removing the stop codon from a cDNA sequence coding for the first protein, then appending the cDNA sequence of the second protein in frame through a method known to a person skilled in the art, such as ligation or overlap extension PCR. The resulting DNA sequence will then be expressed by a cell as a single protein. The protein can be engineered to include the full sequence of both original proteins, or only a portion of either. Optionally, linkers can be included between the components of a fusion protein. In one embodiment, the term “fusion product” relates to a product created by fusing two or more genes, such as a fusion gene comprising a nucleic acid encoding a Fe domain, preferably having a sequence of SEQ ID NO. 7, and comprising a nucleic acid encoding sPD-1, preferably having a sequence of SEQ ID NO. 6, and/or to a product encoded by such fused genes. In one embodiment, an exemplary fusion product of HA-sPD-1 and Fe has a nucleic acid sequence of SEQ ID NO. 8 and/or an amino acid sequence of SEQ ID NO. 13. In one embodiment, a Fe domain or fragment thereof is fused to sPD-1, e.g. HA-sPD-1, which means that a nucleic acid encoding a Fe domain or fragment thereof and a nucleic acid encoding a sPD-1, such as an HA-sPD-1, are fused. In one embodiment, when used in the context of the fusion (F) protein of NDV, the term “fusion protein” does not relate to two or more proteins being joined together, but rather, the term relates to a protein which induces fusion of the viral envelope with the cellular receptor.

The term “high affinity”, as used herein, relates to an increased affinity of a modified protein, such as HA-sPD-1, to its binding partner, such as PD-L1, compared to the affinity of the respective wildtype protein, such as sPD-1, to the respective binding partner. In one embodiment, a “high affinity” version of sPD-1 (HA-sPD-1) has an increased affinity, such as a twofold increased affinity, to its ligands compared to wild type sPD-1. Such high affinity version is, for example, produced via an introduction of a single alanine to leucine substitution at amino acid 132 (A132L) of sPD-1 which results in a higher affinity, such as a 45- and 30-fold higher affinity binding to its ligands, PD-L1 and PD-L2, respectively, or is, for example, a high affinity consensus variant encoding an isoleucine or valine at position 41 resulting in a higher affinity, such as a 40,000-fold higher affinity for PD-L1 compared to wildtype sPD-1. In one embodiment, a high affinity sPD-1 has a K_{d} of <3.2 μM with regard to PD-L1 and <0.1 μM with regard to PD-L2. In one embodiment, a mutation is preferably a point mutation.

SEQ ID NO. 1 represents the amino acid sequence of human PD-1.

SEQ ID NO. 2 represents the amino acid sequence of human PD-1 (soluble).

SEQ ID NO. 3 represents the amino acid sequence of an exemplary high affinity PD-1 (soluble) which is HA-sPD-1-A132L.

SEQ ID NO. 4 represents the amino acid sequence of F3aa-modified fusion protein (F protein) of NDV having amino acid substitution L289A.

SEQ ID NO. 5 represents the amino acid sequence of the HN protein of NDV.

SEQ ID NO. 6 represents the nucleic acid sequence encoding an exemplary high affinity PD-1 (soluble) which is HA-sPD-1-A132L.

SEQ ID NO. 7 represents the nucleic acid sequence of the human IgG1-Fc; CH2 and CH3 domains of the human IgG1 heavy chain and the hinge region.

SEQ ID NO. 8 represents the nucleic acid sequence of an exemplary high affinity soluble PD-1-IgG1-Fc fusion gene.

SEQ ID NO. 9 represents the nucleic acid sequence of an exemplary vector of the present invention comprising a construct VSV-NDV-HA-sPD-1-Fc.

SEQ ID NO. 10 represents the amino acid sequence of a N protein.

SEQ ID NO. 11 represents the amino acid sequence of a P protein.

SEQ ID NO. 12 represents the amino acid sequence of a M protein.

SEQ ID NO. 13 represents the amino acid sequence of a HA-sPD-1-Fc protein.

SEQ ID NO. 14 represents the amino acid sequence of a L protein.

SEQ ID NO. 15 represents the nucleic acid sequence of the construct VSV-NDV-HA-sPD-1-Fc.

SEQ ID NO. 16 represents the total nucleic acid sequence of rVSV-NDV-sPD-1, wherein said sPD-1 is normal sPD-1 without high-affinity modification, and wherein said sequence does not comprise a Fc-encoding sequence.

SEQ ID NO. 17 represents the complete genome of VSV Indiana.

SEQ ID NO. 18 represents the amino acid sequence of VSV Indiana G protein.

SEQ ID NO. 19 represents the nucleic acid sequence of the NDV HN protein.

SEQ ID NO. 20 represents the complete genome of NDV Hitchner B1.

SEQ ID NO. 21 represents the nucleic acid sequence of the NDV F (unmodified) protein.

SEQ ID NO. 22 represents the amino acid sequence of the NDV F (unmodified) protein.

SEQ ID NO. 23 represents the nucleic acid sequence of the NDV F3aa-modified fusion protein with L289A.

SEQ ID NO. 24 represents the nucleic acid sequence of the NDV F3aa-modified fusion protein.

SEQ ID NO. 25 represents the amino acid sequence of the NDV F3aa-modified fusion protein.

SEQ ID NO. 26 represents the nucleic acid sequence of human soluble PD-1 (sPD-1).

### BRIEF DESCRIPTION OF THE FIGURES

The present invention is now further described by reference to the following figures.

All methods mentioned in the figure descriptions below were carried out as described in detail in the examples.

FIG. 1 Shows Interference of the PD-1/PD-L1 Interaction and a Soluble PD-1.

When exhausted T cells come into contact with tumor cells expressing PD-L1, the PD-1 on the T cell engages and becomes inactivated, allowing the tumor cell to evade immune clearance. Local expression of a soluble PD-1 competes with the PD-1 expressed by the T cells for binding to its PD-L1 ligand and interferes with the interaction, enabling the T cell to remain functional and elicit its cytotoxic effector functions against the tumor cell [5].

FIG. 2 Shows the rVSV-NDV Backbone (A) and the rVSV-NDV-sPD-1 Construct of the Present Invention (B).

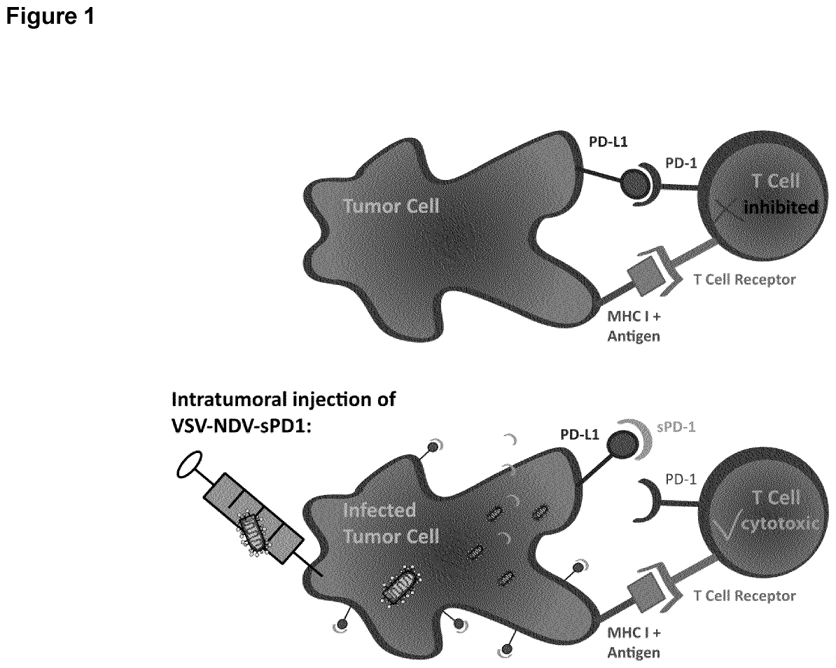

A) Recombinant pseudotyped VSV construct expressing the glycoprotein of NDV (see also WO 2017/198779 Ai). The endogenous glycoprotein of VSV was deleted from a plasmid encoding the full-length VSV genome. The NDV glycoprotein, comprising a modified fusion protein (NDV/F(L289A)) and hemagglutinin-neuraminidase (NDV/HN), was inserted as discrete transcription units between the VSV matrix (M) and large polymerase (L) genes. The respective pseudotyped VSV vector was rescued using an established reverse-genetics system. B) The construct of the present invention comprises a soluble human PD-1 gene, preferably the high affinity soluble human PD-1 gene (HA-sPD-1), fused with a Fe domain of human IgG. The high affinity, soluble human PD-1 gene (HA-sPD-1), fused with the Fe domain of human IgG, was cloned as an additional transcription unit between the NDV attachment protein (HN) and the VSV large polymerase (L) genes. The resultant viral genome is shown.

FIG. 3 shows that rVSV-NDV-HA-sPD-1-Fc replicates with slight attenuation in mouse melanoma cells. The mouse melanoma cell line, B16-OVA, was infected with rVSV-NDV-GFP or rVSV-NDV-HA-sPD-1-Fc at a multiplicity of infection (MOI) of 0.01. After a 1 hour infection, the cells were washed and fresh medium was added to the cells. At various time-points post-infection aliquots of the supernatant were collected for measurements of viral titers by TCID50 assay. Experiments were performed in triplicate, and data are presented as mean +/− standard error of the mean.

FIG. 4 shows that rVSV-NDV-HA-sPD-1-Fc efficiently kills mouse melanoma cells. The mouse melanoma cell line, B16-OVA, was infected with rVSV-NDV-GFP or rVSV-NDV-HA-sPD-1-Fc at a multiplicity of infection (MOI) of 0.01. After a 1 hour infection, the cells were washed and fresh medium was added to the cells. At various time-points post-infection aliquots of the supernatant were collected for LDH assay for cytotoxicity. Experiments were performed in triplicate, and data are presented as mean +/− standard error of the mean.

FIG. 5 shows that rVSV-NDV-HA-sPD-1-Fc causes delayed tumor growth in immune-competent mice bearing syngeneic B16-OVA melanoma cells. Male C57B1/6 mice were implanted with 2.4×10⁵ B16-OVA cells subcutaneously in the flanks. On days 7, 10, and 13 post-tumor implantation, PBS or 10⁷ TCID50 of rVSV-NDV-GFP or rVSV-NDV-HA-sPD-1-Fc was injected into the tumor in a 50 μl volume. Tumor size was monitored daily, and tumor volumes were calculated using the formula: 4/3*PI( )*((L+W)/4)3. Individual tumor growth curves for each treatment are shown. Each curve represents an individual mouse.

FIG. 6 shows that intratumoral injection of rVSV-NDV-HA-sPD-1-Fc significantly prolongs survival of B16-OVA-bearing mice. Male C57B1/6 mice were implanted with 2.4×10⁵ B16-OVA cells subcutaneously in the flanks. On days 7, 10, and 13 post-tumor implantation, PBS or 10⁷ TCID50 of rVSV-NDV-GFP or rVSV-NDV-HA-sPD-1-Fc was injected into the tumor in a 50l volume. Mice were monitored daily and euthanized when tumor diameters reached 1.5 cm or if the skin ruptured due to tumor growth. Survival times post-treatment were plotted as a Kaplan-Meier survival curves and statistical significance was determined by log-rank test. The p value for rVSV-NDV-HA-sPD-1-Fc versus PBS <0.05.

FIG. 7 shows exemplary recombinant VSV-NDV vectors encoding for soluble PD1 (sPD1). The human sPD1 gene was cloned into the VSV-NDV vector as a unique transcription unit between the hemagglutinin-neuraminidase (HN) and large polymerase (L) genes. Additional modifications, including the high affinity (HA) mutation and fusion with a human Fc fragment were additionally engineered. The resulting genomes are shown.

FIG. 8 shows that rVSV-NDV-sPD1 variants have similar growth kinetics and cytotoxic effects in B16 melanoma cells compared to the control rVSV-NDV-GFP. Mouse B16 melanoma cells were infected in vitro with rVSV-NDV-GFP, rVSV-NDV-sPD1, rVSV-NDV-HA-sPD1, rVSV-NDV-sPD1-Fe, or rVSV-NDV-HA-sPD1-Fc at an MOI of 0.01. Top panel: Representative photomicrographs were obtained at 200× magnification at 24- and 48-hours post-infection. Uninfected B16 cells served as a control. Bottom left: Cytotoxicity was quantified at various time-points post-infection using a lactate deyhydrogenase assay (LDH, Promega). Means+standard error of the mean of duplicate experiments are shown. Bottom right: Aliquots of the supernatant harvested at multiple time-points post-infection were subjected to TCID50 analysis for analysis of virus growth kinetics. Means+standard error of the mean of experiments performed in triplicate are shown.

FIG. 9 shows that recombinant sPD1-expressing VSV-NDV vectors produce and secrete human PD1. B16 mouse melanoma cells were infected with VSV-NDV-GFP or the variants of VSV-NDV expressing the soluble human PD1 (VSV-NDV-sPD1, VSV-NDV-HA-sPD1, VSV-NDV-sPD1-Fe, or VSV-NDV-HA-sPD1-Fc) at an MOI of 0.01 or mock-infected. Left: Aliquots of cell lysate and supernatant were collected at various time-points post-infection and subjected to Western blot analysis for human PD1 or GAPDH. Right: Aliquots of supernatant were collected at 24 hours and subjected to ELISA assay for quantification of released human PD1. Means+standard error of the mean are shown.

FIG. 10 shows that a treatment of immune-competent tumor-bearing mice with rVSV-NDV-HA-sPD1-Fc causes improved control of tumor growth and an enrichment of tumor-specific T cells compared to the VSV-NDV control. A) Female C57B1/6 mice were implanted with subcutaneous syngeneic B16 melanoma cells in contralateral flanks. PBS, 10⁷ TCID50 of rVSV-NDV, or rVSV-NDV-HA-sPD1-Fc was injected into the tumor on the right flank, while left flank tumors remained untreated. Tumors volumes were monitored daily, and blood was collected on day 15. B) Circulating OVA-specific T cells were quantified by flow cytometry using an OVA-specific tetramer to stain peripheral blood mononuclear cells (PBMCs) obtained from blood. Individual data points, means, and standard error of the mean are shown. C) The tumor volume was significantly decreased in the rVSV-NDV-HA-sPD1-Fc group compared to the rVSV-NDV and PBS groups.

In the following, reference is made to the examples, which are given to illustrate, not to limit the present invention.

### EXAMPLES

### Example 1: Preparation of the rVSV-NDV-sPD-1 Virus

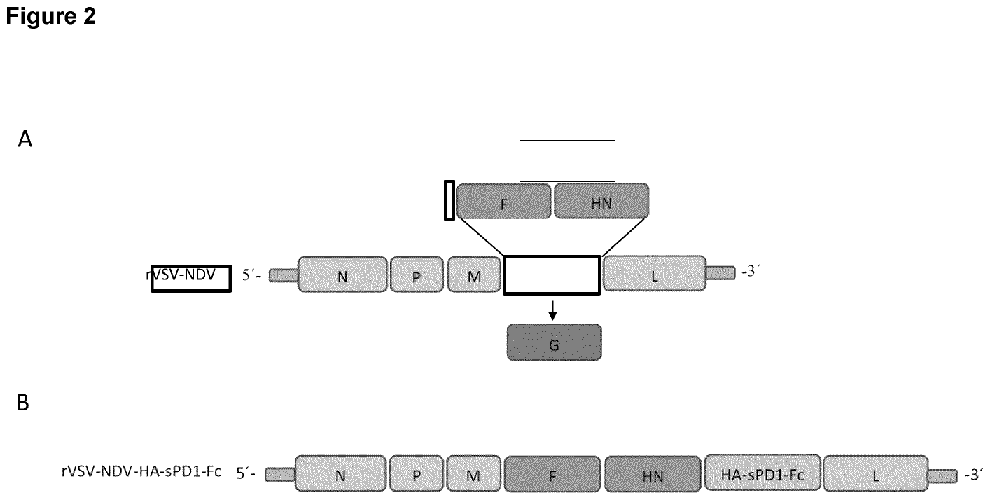

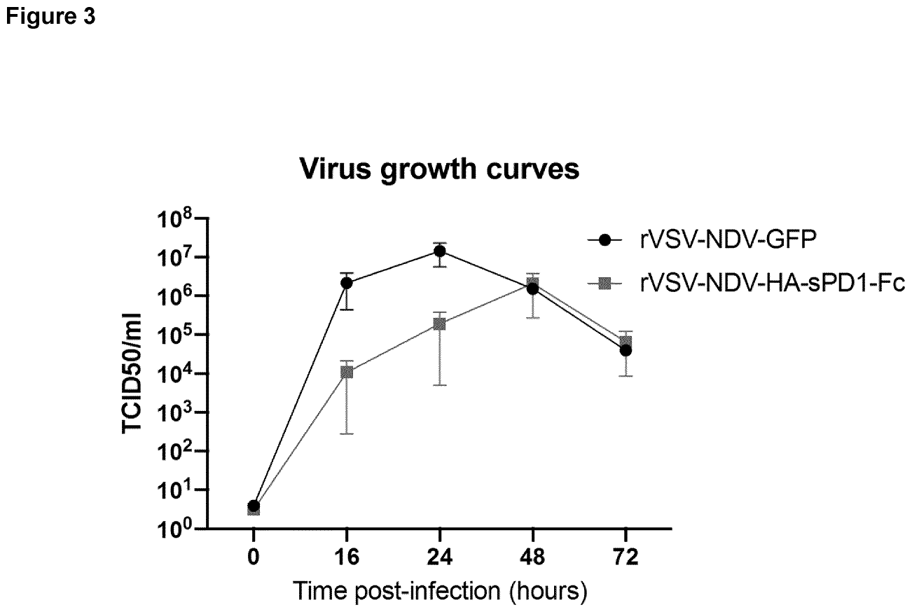

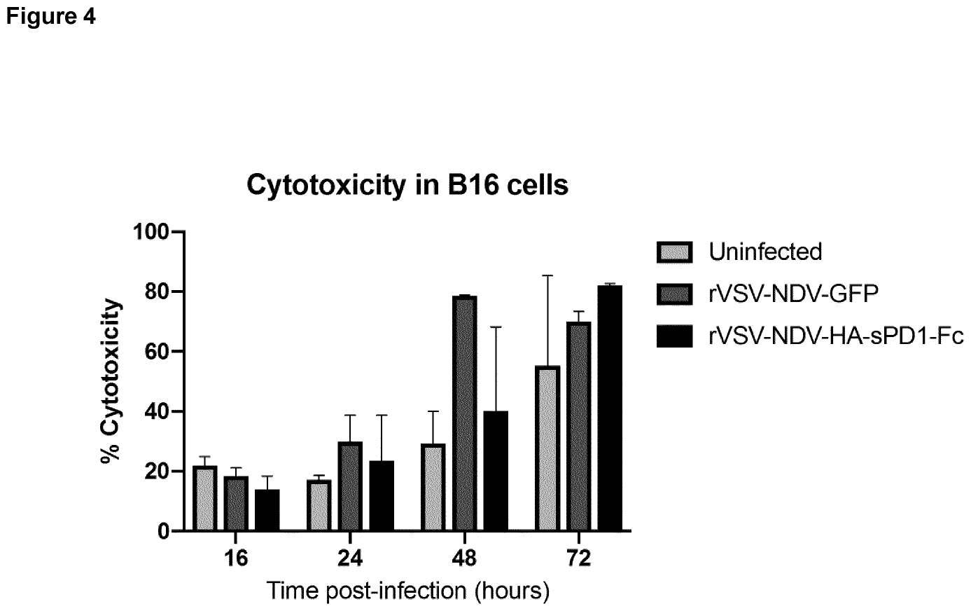

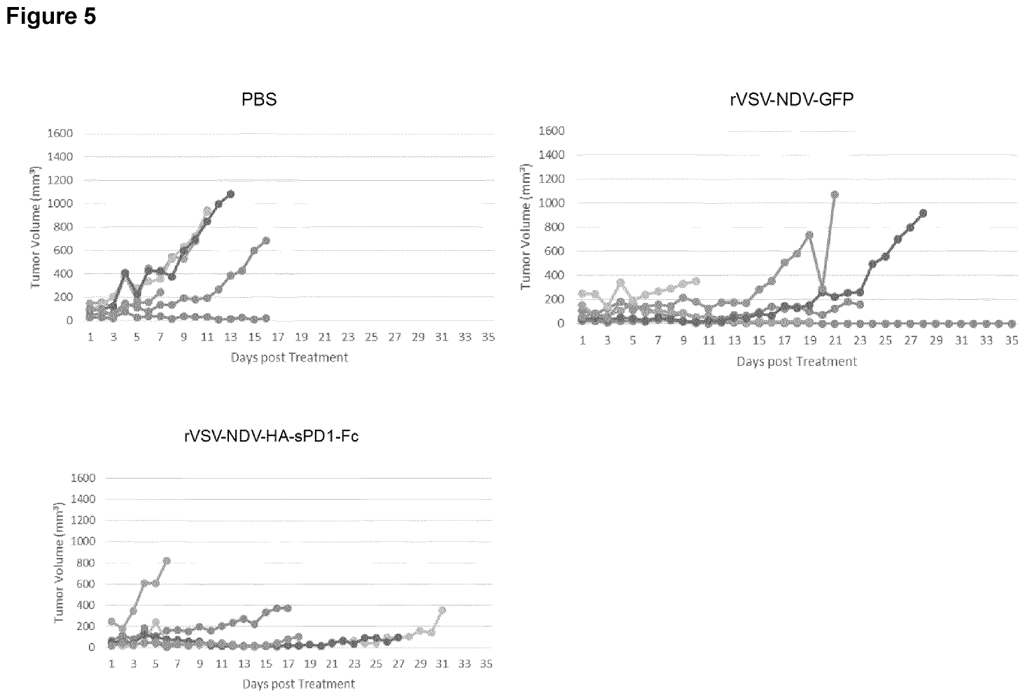

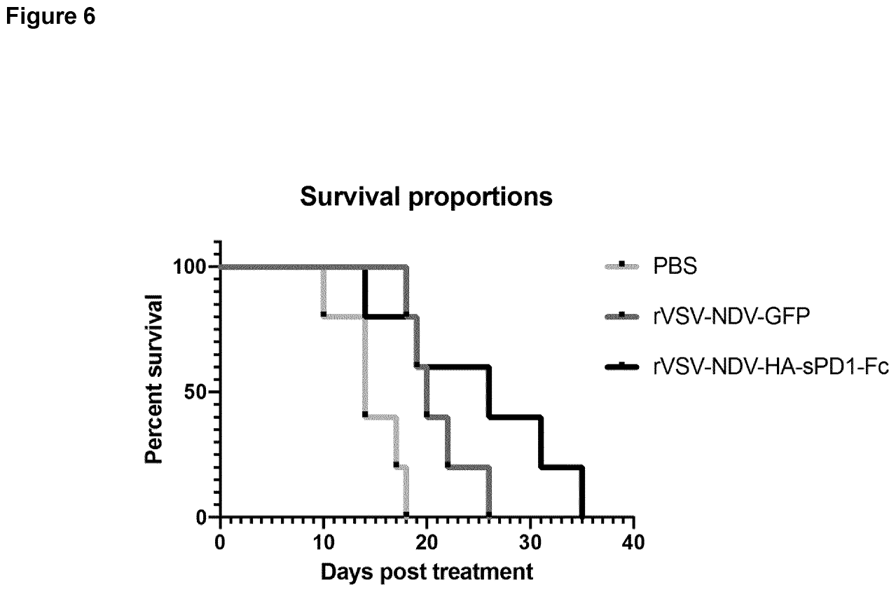

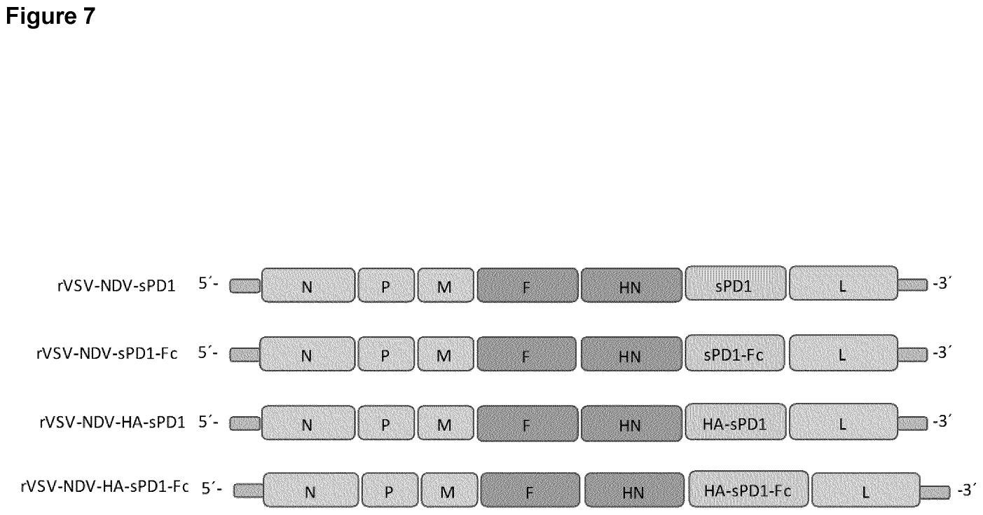

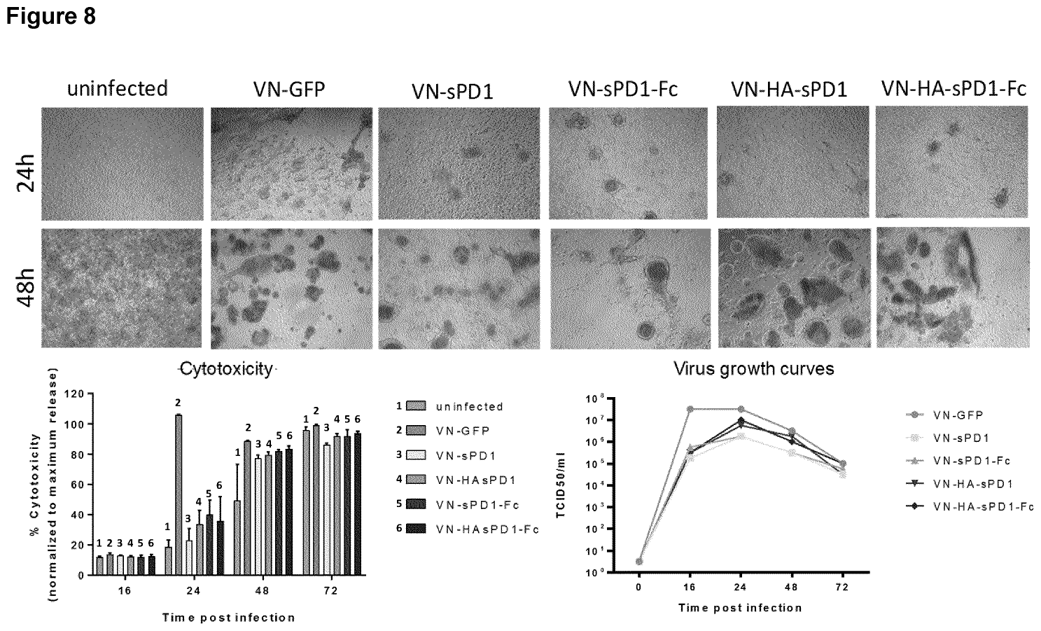

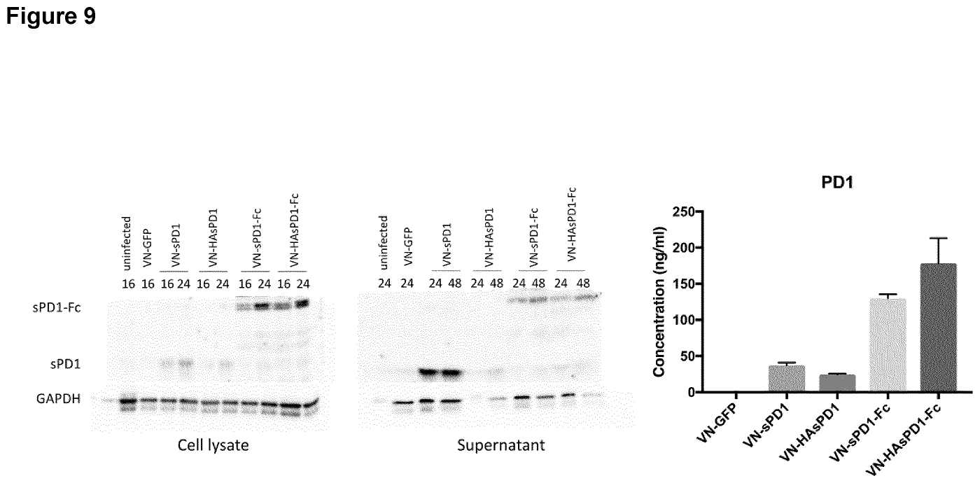

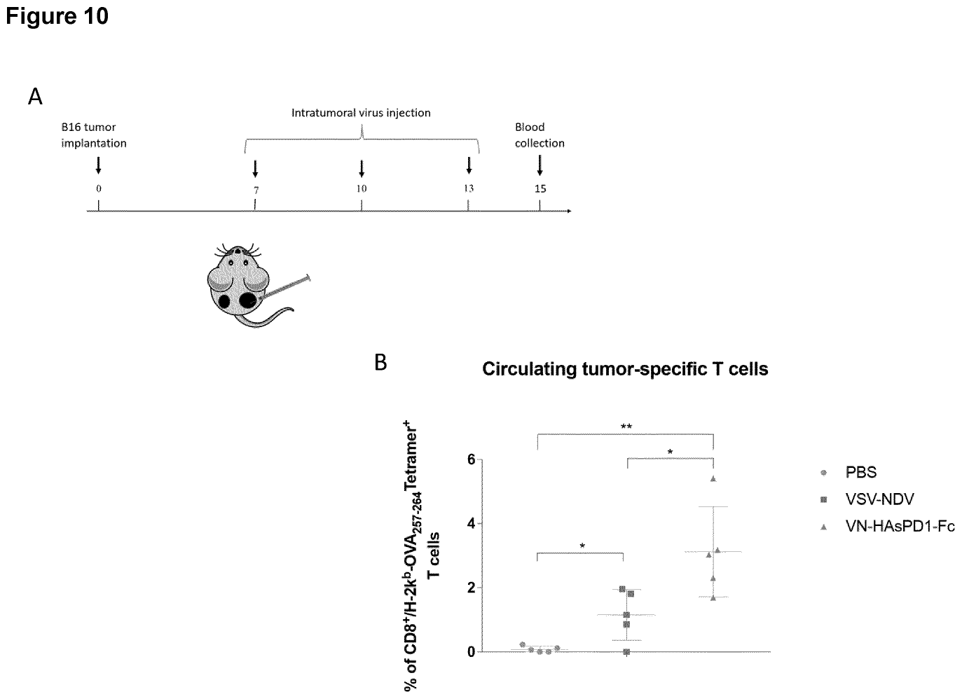

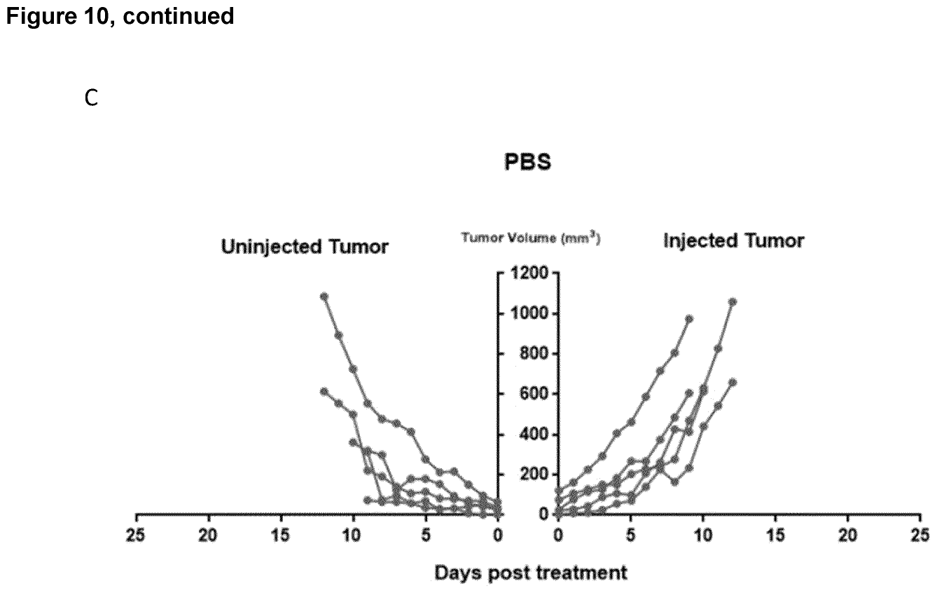

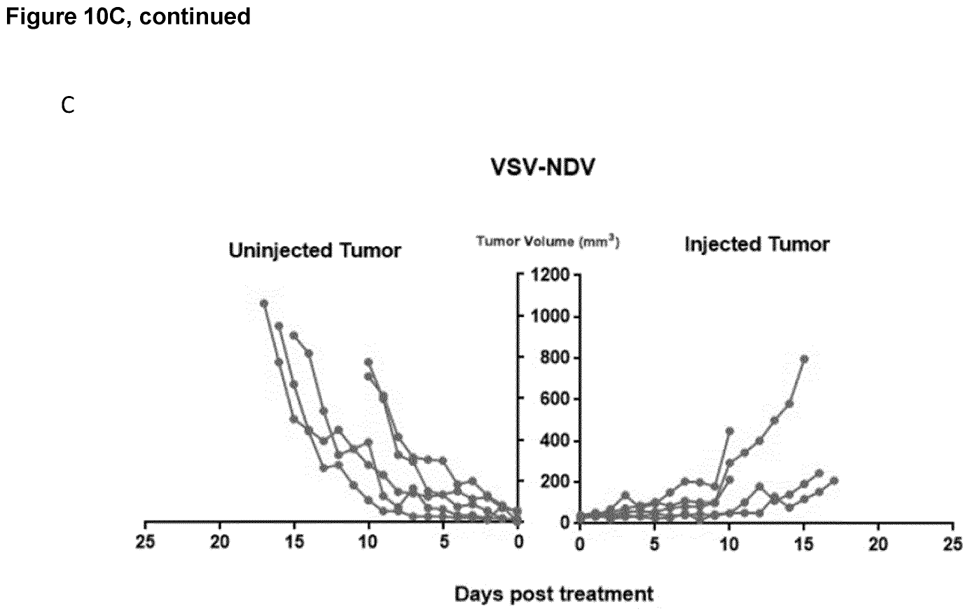

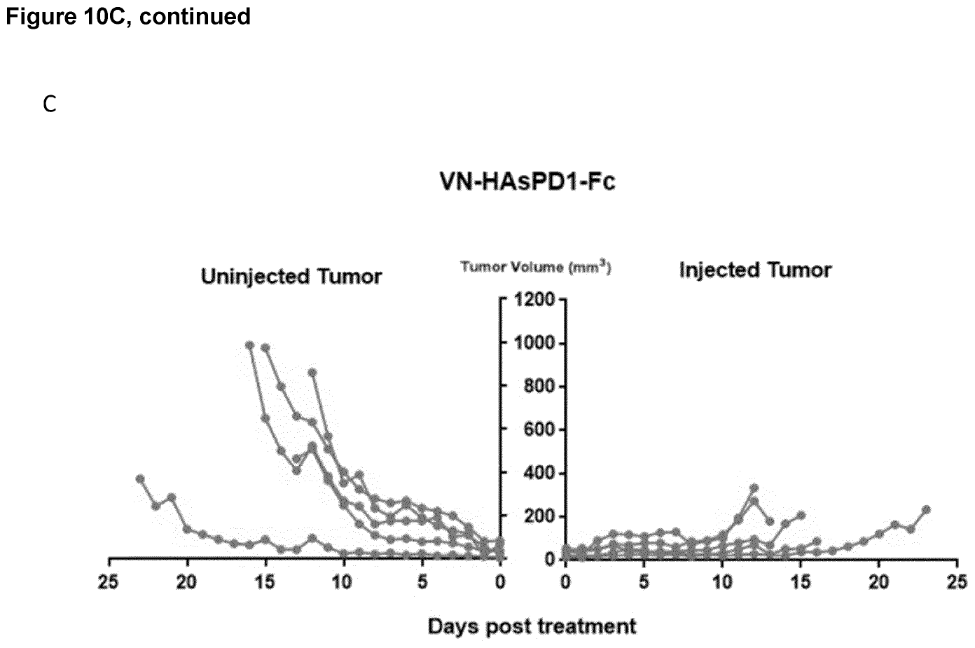

In order to engineer the rVSV-NDV-sPD-1 virus, the signaling and extracellular domains of PD-1 were first amplified by RT-PCR from human PBMCs. The RT-PCR product generated served as a template for a round of overlapping PCR to introduce the A132L mutation for high affinity, which generated 2 overlapping PCR fragments containing the mutated base pairs. These fragments were annealed and subjected to a final elongation step to produce the full-length HA-sPD-1 gene. The HA-sPD-1 fragment was cloned into pFUSE-hIgG1-Fc1 in order to create a fusion gene of the HA sPD-1 with the human Fc fragment. In order to insert the HA-sPD-1-Fc construct into rVSV-NDV, the appropriate restriction sites were introduced using forward and reverse oligonucleotides and amplified by PCR. Finally, the insert was ligated into the full-length VSV-NDV genome as an additional transcription unit via the multi-cloning site between the NDV-HN and the VSV-L genes. The virus construct is depicted in FIG. 2B. The corresponding infectious recombinant virus was rescued using an established method of reverse genetics.

### Example 2: Characterization of the rVSV-NDV-sPD-1 Virus

Characterization of the recombinant VSV-NDV-HA-sPD-1-Fc virus was carried out in the mouse melanoma cell line B16-OVA. Growth kinetics of the virus were compared to the parental rVSV-NDV virus and revealed a slight attenuation attributed to the expression of the transgene, and a peak in replication was reached at approximately 48-hours post-infection at a multiplicity of infection (MOI) of 0.01 (FIG. 3). In line with these findings, cytotoxicity assays in the same cells revealed a slight delay in tumor cell killing by infection with rVSV-NDV-HA-sPD-1-Fc, but nearly complete killing of the cell monolayer by 72-hours post-infection (FIG. 4). Accordingly, the VSV-NDV-HA-sPD-1-Fc virus provides enhanced efficiency after 72 hours post-infection compared to the virus without HA-sPD-1-Fc.

### Example 3: Anticancer Effect of the rVSV-NDV-sPD-1 Virus In Vivo

In order to determine in vivo efficacy, experiments were performed in immune-competent C57B1/6 mice bearing subcutaneous B16-OVA tumors in their flanks. On days 7, 10, and 13 after tumor implantation, PBS or 10⁷ TCID50 of rVSV-NDV-GFP or rVSV-NDV-HA-sPD-1-Fc was injected into the tumor in a 50 μl volume. Tumors were measured daily, and tumor volumes were calculated using the following formula: V=4/3*PI( )*((L+W)/4)3. The data reveal a significant delay in tumor growth in those mice treated with rVSV-NDV-GFP, which was even more pronounced in mice receiving rVSV-NDV-HA-sPD-1-Fe, compared to PBS (FIG. 5).

### Example 4: Increased Survival of Mice Treated with rVSV-NDV-HA-sPD-1-Fc

In a survival study, mice were euthanized at humane endpoints when tumors reached a diameter of 1.5 cm or if tumor growth led to skin ruptures. Survival times with respect to the first treatment dose were plotted and revealed a significant survival prolongation of mice treated with rVSV-NDV-HA-sPD-1-Fc compared to PBS, with a median survival time of 26 versus 14 days, respectively (FIG. 6). The light grey line represents survival time after treatment with PBS; medium grey represents that of rVSV-NDV-GFP treatment; black represents that of rVSV-NDV-HA-sPD-1-Fc treatment. The data demonstrate that treatment with rVSV-NDV-HA-sPD-1-Fc results in substantially delayed tumor growth, resulting in prolonged survival, compared to treatment with buffer or the parental rVSV-NDV virus.

### Example 5: Recombinant VSV-NDV Vectors Encoding for Soluble PD1 (sPD1)

A panel of VSV-NDV recombinant vectors encoding for the soluble PD1, either with the human Fc fragment (sPD1-Fc) or without (sPD1) and with the high affinity mutation (HA-sPD1) or without, were engineered and rescued by the established reverse genetics system (FIG. 7). These rVSV-NDV-sPD1 variants were fully characterized in comparison with the control rVSV-NDV vector in vitro, and the rVSV-NDV-HA-sPD1-Fc vector was selected for further characterization in vivo in an immune-competent model of mouse melanoma.

### Example 6: rVSV-NDV-sPD1 Variants Replicate Well and Cause Cytotoxicity in B16 Mouse Melanoma Cells

In order to characterize the panel of rVSV-NDV-sPD1 variants in vitro, a B16 mouse melanoma cell line was chosen as representative. B16 cells were infected with the various sPD1 variants or the VSV-NDV-GFP control virus at a multiplicity of infection (MOI) of 0.01. Cells were examined microscopically at various time-points post infection in order to visualize cytotoxic effects. Additionally, samples of the supernatant were collected for analysis of cell viability by lactate dehydrogenase (LDH) assay and quantification of virus replication by tissue culture infectious dose 50 (TCID50) assay. These data revealed no substantial changes in virus replication or tumor cell killing kinetics among the sPD1 variants or in comparison to rVSV-NDV (FIG. 8).

### Example 7: rVSV-NDV-sPD1 Variants Produce and Release Soluble Human PD1 In Vitro

In order to confirm that the recombinant constructs produce and release the soluble human PD1, Western blots and ELISA assays were performed. B16 cells were infected with the sPD1-expressing VSV-NDV constructs or the control VSV-NDV-GFP at an MOI of 0.01 or left uninfected, and lysates and supernatants were collected at various time-points post-infection and subjected to Western blot analysis for sPD1. Additionally, GAPDH was analyzed to control for protein loading. As expected, the sPD1 virus-infected samples produced a band at the expected size, while the viruses expressing the sPD1-Fc fusion protein had a substantial shift in size corresponding to the additive size of the sPD1 and the Fc fragment (FIG. 9, left panel). Additional samples of supernatant collected at 24 hours were subjected to an ELISA assay to quantify the amount of PD1 secreted into the supernatant. As expected, only samples from cells infected with VSV-NDV expressing sPD1 produced detectable levels of PD1, while those expressing sPD1 fused with the Fc fragment (sPD1-Fc) had even higher levels than those without, supporting the hypothesis that the presence of the Fc fragment stabilizes the extracellular sPD1 (FIG. 9, right panel).

### Example 8: In Vivo rVSV-NDV-HA-sPD1-Fc Treatment of Syngeneic Mouse Melanoma Results in Enhanced Tumor-Specific T Cell Responses and Delayed Tumor Growth

For in vivo analysis of VSV-NDV-mediated expression of sPD1, the inventors focused on the HA-sPD1-Fc variant, due to a potential benefit of high affinity binding via the HA mutation and stability in blood afforded by fusion of sPD1 with the Fc fragment. The inventors utilized a subcutaneous model of B16 melanoma, in which tumors were implanted into contralateral flanks, and PBS, rVSV-NDV, or rVSV-NDV-HA-sPD1-Fc at a dose of 10⁷ TCID50 was injected intratumorally into the lesion on the right flank on day 7, 10, and 13 after tumor-implantation (FIG. 10A). Peripheral blood mononuclear cells (PBMCs) were isolated from blood collected on day 15 and subjected to FACS analysis of CD8+ T cells specific for the model antigen, ovalbumin, that is expressed by the B16 cells. While VSV-NDV treatment resulted in a statistically significant increase in OVA-specific T cells compared to PBS, this effect was even further increased in the mice treated with rVSV-NDV-HA-sPD1-Fc (FIG. 10B).

Furthermore, analysis of tumor growth in both, the injected and contralateral uninjected lesions, revealed delayed growth in rVSV-NDV-HA-sPD1-Fc-treated animals compared to rVSV-NDV- or PBS-treated (FIG. 10C). These results indicate that rVSV-NDV-HA-sPD1-Fc allows for enhanced T cell activation, leading to immune-mediated abscopal effects.

### REFERENCES

[1] Bartee MY, Dunlap KM, Bartee E. Tumor-Localized Secretion of Soluble PD1 Enhances Oncolytic Virotherapy. Cancer Res. 2017; 77(11):2952-63.
[2] Lazar-Molnar E, Scandiuzzi L, Basu I, Quinn T, Sylvestre E, Palmieri E, et al. Structure-guided development of a high-affinity human Programmed Cell Death-1: Implications for tumor immunotherapy. EBioMedicine. 2017; 17:30-44.
[3] Maute R L, Gordon S R, Mayer A T, McCracken M N, Natarajan A, et al. PD-1 variants for immunotherapy and PET imaging. PNAS. Nov 2015, 112 (47) E6506-E6514; DOI: 10.1073/pnas.1519623112.
[4] Altomonte, J., S. Marozin, et al. (2010). “Engineered newcastle disease virus as an improved oncolytic agent against hepatocellular carcinoma.” Mol Ther 18(2): 275-284.
[5] Malini Guha, The Pharmaceutical Journal (2014).

The features of the present invention disclosed in the specification, the claims, and/or in the accompanying figures may, both separately and in any combination thereof, be material for realizing the invention in various forms thereof.

1. A recombinant oncolytic virus,
comprising a vesicular stomatitis virus (VSV),
wherein the glycoprotein (G protein) of VSV is deleted, and which comprises
a modified fusion protein (F protein) of Newcastle disease virus (NDV), and
the hemagglutinin neuraminidase (HN) protein of NDV,
further comprising soluble PD-1 (sPD-1).

2. The recombinant oncolytic virus according to claim 1, wherein said recombinant virus further comprises a Fc domain or a fragment thereof.

3. The recombinant oncolytic virus according to claim 1, wherein said Fe domain or fragment thereof is fused to said sPD-1.

4. The recombinant oncolytic virus according to claim 1, wherein said sPD-1 is a high affinity sPD-1 (HA-sPD-1).

5. The recombinant oncolytic virus according to claim 1, wherein said sPD-1 comprises a mutation at a position selected from 132 and 41 of SEQ ID NO: 2.

6. The recombinant oncolytic virus according to claim 1, wherein said sPD-1 is HA-sPD-1-A132L having a sequence of SEQ ID NO: 3.

7. The recombinant oncolytic virus according to claim 1, wherein the modified fusion protein (F protein) of NDV is the F3aa-modified F protein having a sequence of SEQ ID NO-_25,
and/or comprises at least one amino acid substitution in the protease cleavage site;
and/or wherein the modified fusion protein (F protein) of NDV is the F3aa-modified F protein with an amino acid substitution L289A having SEQ ID NO. 4;
and/or wherein the G protein of VSV is replaced by the modified fusion protein having a sequence of SEQ ID NO: 4 and HN protein of NDV having a sequence of SEQ ID NO-_5.

8. A nucleic acid encoding a recombinant oncolytic virus of claim 1.

9. The nucleic acid according to claim 8, comprising a nucleic acid encoding a Fe domain or fragment thereof, which is fused to a nucleic acid encoding said sPD-1.

10. A vector comprising a nucleic acid of claim 8.

11. The nucleic acid according to claim 8, comprising the nucleotide sequence of SEQ ID NO: 9 or 15,
or a nucleotide sequence having at least 60% sequence identity to the nucleotide sequence of SEQ ID NO: 9 or 15,
or comprising the nucleotide sequence of SEQ ID NO: 16,
or a nucleotide sequence having at least 60% sequence identity to the nucleotide sequence of SEQ ID NO: 16.

12. A pharmaceutical composition, comprising
the recombinant oncolytic virus of claim 1, or a nucleic acid encoding the recombinant oncolytic virus of claim 1.

13. The pharmaceutical composition according to claim 12, formulated for any of systemic delivery, tumor injection, intravenous administration, and intra-arterial administration, and/or for an intradermal, subcutaneous, intramuscular, intravenous, intraosseous, intraperitoneal, intrathecal, epidural, intracardiac, intraarticular, intracavernous, intracerebral, intracerebroventricular, or intravitreal injection.

14. (canceled)

15. A method for the prevention and/or treatment of cancer wherein said method comprises administering to a subject in need of such prevention or treatment the recombinant oncolytic virus of claim 1, or a nucleic acid encoding the virus of claim 1.

16. A method for gene delivery, imaging of virus biodistribution, and/or for tumor detection wherein said method comprises the use of the oncolytic virus of claim 1, or a nucleic acid encoding the oncolytic virus of claim 1.

17. The method according to claim 15, wherein the cancer is selected from hepatocellular carcinoma, pancreatic cancer, and melanoma.

18. The recombinant oncolytic virus according to claim 2, wherein said sPD-1 is a high affinity sPD-1 (HA-sPD-1), having an affinity to its ligands PD-L1 and PD-L2 that is at least 2-fold higher than an affinity of a wildtype sPD-1 to said ligands, and/or has an affinity with a K_{d} of <3.2 μM with regard to PD-L1 and/or <0.1 μM with regard to PD-L2.

19. The recombinant oncolytic virus according to claim 5, wherein said sPD-1 comprises a mutation at a position selected from 132 and 41 of SEQ ID NO: 2, wherein said mutation is selected from A132L, L41I, and L41V.

20. The recombinant oncolytic virus according to claim 7, wherein the modified fusion protein (F protein) of NDV is a F3aa-modified F protein having an L289A amino acid substitution.

21. The nucleic acid according to claim 8, comprising a nucleic acid encoding a Fc domain having a sequence of SEQ ID NO: 7, which is fused to a nucleic acid encoding said sPD-1 having a sequence of SEQ ID NO: 6.


SEQUENCE LISTING (26 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 170 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 3 - protein, 170 aa, Artificial Sequence
  High Affinity PD1 (soluble)
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLV

SEQ ID NO: 4 - protein, 553 aa, Artificial Sequence
  NDV F3aa-modified fusion protein with L289A (amino acid sequence)
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGRRQRRFIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTAPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 5 - protein, 577 aa, Newcastle disease virus
MDRAVSQVALENDEREAKNTWRLIFRIAILFLTVVTLAISVASLLYSMGASTPSDLVGIPTRISRAEEKITSTLGSNQDVVDRIYKQVALESPLALLNTETTIMNAITSLSYQINGAANNSGWGAPIHDPDYIGGIGKELIVDDASDVTSFYPSAFQEHLNFIPAPTTGSGCTRIPSFDMSATHYCYTHNVILSGCRDHSHSHQYLALGVLRTSATGRVFFSTLRSINLDDTQNRKSCSVSATPLGCDMLCSKATETEEEDYNSAVPTRMVHGRLGFDGQYHEKDLDVTTLFGDWVANYPGVGGGSFIDSRVWFSVYGGLKPNTPSDTVQEGKYVIYKRYNDTCPDEQDYQIRMAKSSYKPGRFGGKRIQQAILSIKVSTSLGEDPVLTVPPNTVTLMGAEGRILTVGTSHFLYQRGSSYFSPALLYPMTVSDKTATLHSPYTFNAFTRPGSIPCQASARCPNSCVTGVYTDPYPLIFYRNHTLRGVFGTMLDGEQARLNPASAVFDSTSRSRITRVSSSSIKAAYTTSTCFKVVKTNKTYCLSIAEISNTLFGEFRIVPLLVEILKDDGVREARSG

SEQ ID NO: 6 - DNA, 510 bp, Artificial Sequence
  High affinity PD1 (soluble) nucleic acid sequence
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctgcagatcaaagagagcctgcgggcagagctcagggtgacagagagaagggcagaagtgcccacagcccaccccagcccctcacccaggccagccggccagttccaaaccctggtg

SEQ ID NO: 7 - DNA, 684 bp, Artificial Sequence
  Human IgG1-Fc; CH2 and CH3 domains of the human IgG1 heavy chain and the hinge region
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 8 - DNA, 1200 bp, Artificial Sequence
  High affinity soluble PD1-IgG1-Fc fusion gene
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctgcagatcaaagagagcctgcgggcagagctcagggtgacagagagaagggcagaagtgcccacagcccaccccagcccctcacccaggccagccggccagttccaaaccctggtgctcgaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatga

SEQ ID NO: 9 - DNA, 17346 bp, Artificial Sequence
  vector nucleic acid sequence
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactataggacgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcgagatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgagctagcttaattaatatgaaaaaaactaacagatatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctaggtttaaacagctaggcgcgcctatgaaaaaaactaacagcaatcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctgcagatcaaagagagcctgcgggcagagctcagggtgacagagagaagggcagaagtgcccacagcccaccccagcccctcacccaggccagccggccagttccaaaccctggtgctcgaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgacctagggctagcggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 10 - protein, 422 aa, Artificial Sequence
  N protein
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 11 - protein, 265 aa, Artificial Sequence
  P protein
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 12 - protein, 229 aa, Artificial Sequence
  M protein
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 13 - protein, 399 aa, Artificial Sequence
  sPD1-Fc protein
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKLQIKESLRAELRVTERRAEVPTAHPSPSPRPAGQFQTLVLEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 2109 aa, Artificial Sequence
  L protein
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 15 - DNA, 14086 bp, Artificial Sequence
  VSV-NDV-HA-sPD1-Fc (nucleotide sequence)
atgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcgagatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgagctagcttaattaatatgaaaaaaactaacagatatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctaggtttaaacagctaggcgcgcctatgaaaaaaactaacagcaatcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctgcagatcaaagagagcctgcgggcagagctcagggtgacagagagaagggcagaagtgcccacagcccaccccagcccctcacccaggccagccggccagttccaaaccctggtgctcgaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgacctagggctagcggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaa

SEQ ID NO: 16 - DNA, 16659 bp, Artificial Sequence
  Total nucleic acid sequence of rVSV-NDV-sPD1
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactataggacgaagacaaacaaaccattattatcattaaaaggctcaggagaaactttaacagtaatcaaaatgtctgttacagtcaagagaatcattgacaacacagtcgtagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggcttgtatgcaccagatccagaagctgagcaagttgaaggctttatacaggggcctttagatgactatgcagatgaggaagtggatgttgtatttacttcggactggaaacagcctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacgctcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggactctatcggccacttcaaatgagctagtctaacttctagcttctgaacaatccccggtttactcagtctcccctaattccagcctctcgaacaactaatatcctgtcttttctatccctatgaaaaaaactaacagagatcgatctgtttacgcgtcgagatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgagctagcttaattaatatgaaaaaaactaacagatatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctaggtttaaacagctaggcgcgcctatgaaaaaaactaacagcaatcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgtaacctagggctagcggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaacctgaattctcctctaattagtgatgatattgacaatttaatcaggaaattcaattctcttccaattccctcgatgtgggatagtaagaactgggatggagttcttgagatgttaacgtcatgtcaagccaatcccatcccaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggagtatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaatttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgtgggtcggcatggcatctccacctcctcgcggtccgacctgggcatccgaaggaggacgtcgtccactcggatggctaagggaggggcccccgcggggctgctaacaaagcccgaaaggaagctgagttggctgctgccaccgctgagcaataactagcataaccccttggggcctctaaacgggtcttgaggggttttttgctgaaaggaggaactatatccggatcgagacctcgatactagtgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 17 - DNA, 11161 bp, Vesicular stomatitis Indiana virus
acgaagacaaacaaaccattattatcattaaaaggctcaggagaaactttaacagtaatcaaaatgtctgttacagtcaagagaatcattgacaacacagtcatagttccaaaacttcctgcaaatgaggatccagtggaatacccggcagattacttcagaaaatcaaaggagattcctctttacatcaatactacaaaaagtttgtcagatctaagaggatatgtctaccaaggcctcaaatccggaaatgtatcaatcatacatgtcaacagctacttgtatggagcattaaaggacatccggggtaagttggataaagattggtcaagtttcggaataaacatcgggaaagcaggggatacaatcggaatatttgaccttgtatccttgaaagccctggacggcgtacttccagatggagtatcggatgcttccagaaccagcgcagatgacaaatggttgcctttgtatctacttggcttatacagagtgggcagaacacaaatgcctgaatacagaaaaaagctcatggatgggctgacaaatcaatgcaaaatgatcaatgaacagtttgaacctcttgtgccagaaggtcgtgacatttttgatgtgtggggaaatgacagtaattacacaaaaattgtcgctgcagtggacatgttcttccacatgttcaaaaaacatgaatgtgcctcgttcagatacggaactattgtttccagattcaaagattgtgctgcattggcaacatttggacacctctgcaaaataaccggaatgtctacagaagatgtaacgacctggatcttgaaccgagaagttgcagatgaaatggtccaaatgatgcttccaggccaagaaattgacaaggccgattcatacatgccttatttgatcgactttggattgtcttctaagtctccatattcttccgtcaaaaaccctgccttccacttctgggggcaattgacagctcttctgctcagatccaccagagcaaggaatgcccgacagcctgatgacattgagtatacatctcttactacagcaggtttgttgtacgcttatgcagtaggatcctctgccgacttggcacaacagttttgtgttggagataacaaatacactccagatgatagtaccggaggattgacgactaatgcaccgccacaaggcagagatgtggtcgaatggctcggatggtttgaagatcaaaacagaaaaccgactcctgatatgatgcagtatgcgaaaagagcagtcatgtcactgcaaggcctaagagagaagacaattggcaagtatgctaagtcagaatttgacaaatgaccctataattctcagatcacctattatatattatgctacatatgaaaaaaactaacagatatcatggataatctcacaaaagttcgtgagtatctcaagtcctattctcgtctggatcaggcggtaggagagatagatgagatcgaagcacaacgagctgaaaagtccaattatgagttgttccaagaggatggagtggaagagcatactaagccctcttattttcaggcagcagatgattctgacacagaatctgaaccagaaattgaagacaatcaaggtttgtatgcacaggatccagaagctgagcaagttgaaggctttatacaggggcctttagatgactatgcagatgaggaagtggatgttgtatttacttcggactggaaaccacctgagcttgaatctgacgagcatggaaagaccttacggttgacatcgccagagggtttaagtggagagcagaaatcccagtggctttcgacgattaaagcagtcgtgcaaagtgccaaatactggaatctggcagagtgcacatttgaagcatcgggagaaggggtcattatgaaggagcgccagataactccggatgtatataaggtcactccagtgatgaacacacatccgtcccaatcagaagcagtatcagatgtttggtctctctcaaagacatccatgactttccaacccaagaaagcaagtcttcagcctctcaccatatccttggatgaattgttctcatctagaggagagttcatctctgtcggaggtgacggacgaatgtctcataaagaggccatcctgctcggcctgagatacaaaaagttgtacaatcaggcgagagtcaaatattctctgtagactatgaaaaaaagtaacagatatcacgatctaagtgttatcccaatccattcatcatgagttccttaaagaagattctcggtctgaaggggaaaggtaagaaatctaagaaattagggatcgcaccacccccttatgaagaggacactagcatggagtatgctccgagcgctccaattgacaaatcctattttggagttgacgagatggacacctatgatccgaatcaattaagatatgagaaattcttctttacagtgaaaatgacggttagatctaatcgtccgttcagaacatactcagatgtggcagccgctgtatcccattgggatcacatgtacatcggaatggcagggaaacgtcccttctacaaaatcttggcttttttgggttcttctaatctaaaggccactccagcggtattggcagatcaaggtcaaccagagtatcacactcactgcgaaggcagggcttatttgccacataggatggggaagacccctcccatgctcaatgtaccagagcacttcagaagaccattcaatataggtctttacaagggaacgattgagctcacaatgaccatctacgatgatgagtcactggaagcagctcctatgatctgggatcatttcaattcttccaaattttctgatttcagagagaaggccttaatgtttggcctgattgtcgagaaaaaggcatctggagcgtgggtcctggattctatcagccacttcaaatgagctagtctaacttctagcttctgaacaatccccggtttactcagtctctcctaattccagcctctcgaacaactaatatcctgtcttttctatccctatgaaaaaaactaacagagatcgatctgtttccttgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaactaacagcaatcatggaagtccacgattttgagaccgacgagttcaatgatttcaatgaagatgactatgccacaagagaattcctgaatcccgatgagcgcatgacgtacttgaatcatgctgattacaatttgaattctcctctaattagtgatgatattgacaatttgatcaggaaattcaattctcttccgattccctcgatgtgggatagtaagaactgggatggagttcttgagatgttaacatcatgtcaagccaatcccatctcaacatctcagatgcataaatggatgggaagttggttaatgtctgataatcatgatgccagtcaagggtatagttttttacatgaagtggacaaagaggcagaaataacatttgacgtggtggagaccttcatccgcggctggggcaacaaaccaattgaatacatcaaaaaggaaagatggactgactcattcaaaattctcgcttatttgtgtcaaaagtttttggacttacacaagttgacattaatcttaaatgctgtctctgaggtggaattgctcaacttggcgaggactttcaaaggcaaagtcagaagaagttctcatggaacgaacatatgcaggattagggttcccagcttgggtcctacttttatttcagaaggatgggcttacttcaagaaacttgatattctaatggaccgaaactttctgttaatggtcaaagatgtgattatagggaggatgcaaacggtgctatccatggtatgtagaatagacaacctgttctcagagcaagacatcttctcccttctaaatatctacagaattggagataaaattgtggagaggcagggaaatttttcttatgacttgattaaaatggtggaaccgatatgcaacttgaagctgatgaaattagcaagagaatcaaggcctttagtcccacaattccctcattttgaaaatcatatcaagacttctgttgatgaaggggcaaaaattgaccgaggtataagattcctccatgatcagataatgagtgtgaaaacagtggatctcacactggtgatttatggatcgttcagacattggggtcatccttttatagattattacactggactagaaaaattacattcccaagtaaccatgaagaaagatattgatgtgtcatatgcaaaagcacttgcaagtgatttagctcggattgttctatttcaacagttcaatgatcataaaaagtggttcgtgaatggagacttgctccctcatgatcatccctttaaaagtcatgttaaagaaaatacatggcccacagctgctcaagttcaagattttggagataaatggcatgaacttccgctgattaaatgttttgaaatacccgacttactagacccatcgataatatactctgacaaaagtcattcaatgaataggtcagaggtgttgaaacatgtccgaatgaatccgaacactcctatccctagtaaaaaggtgttgcagactatgttggacacaaaggctaccaattggaaagaatttcttaaagagattgatgagaagggcttagatgatgatgatctaattattggtcttaaaggaaaggagagggaactgaagttggcaggtagatttttctccctaatgtcttggaaattgcgagaatactttgtaattaccgaatatttgataaagactcatttcgtccctatgtttaaaggcctgacaatggcggacgatctaactgcagtcattaaaaagatgttagattcctcatccggccaaggattgaagtcatatgaggcaatttgcatagccaatcacattgattacgaaaaatggaataaccaccaaaggaagttatcaaacggcccagtgttccgagttatgggccagttcttaggttatccatccttaatcgagagaactcatgaattttttgagaaaagtcttatatactacaatggaagaccagacttgatgcgtgttcacaacaacacactgatcaattcaacctcccaacgagtttgttggcaaggacaagagggtggactggaaggtctacggcaaaaaggatggactatcctcaatctactggttattcaaagagaggctaaaatcagaaacactgctgtcaaagtcttggcacaaggtgataatcaagttatttgcacacagtataaaacgaagaaatcgagaaacgttgtagaattacagggtgctctcaatcaaatggtttctaataatgagaaaattatgactgcaatcaaaatagggacagggaagttaggacttttgataaatgacgatgagactatgcaatctgcagattacttgaattatggaaaaataccgattttccgtggagtgattagagggttagagaccaagagatggtcacgagtgacttgtgtcaccaatgaccaaatacccacttgtgctaatataatgagctcagtttccacaaatgctctcaccgtagctcattttgctgagaacccaatcaatgccatgatacagtacaattattttgggacatttgctagactcttgttgatgatgcatgatcctgctcttcgtcaatcattgtatgaagttcaagataagataccgggcttgcacagttctactttcaaatacgccatgttgtatttggacccttccattggaggagtgtcgggcatgtctttgtccaggtttttgattagagccttcccagatcccgtaacagaaagtctctcattctggagattcatccatgtacatgctcgaagtgagcatctgaaggagatgagtgcagtatttggaaaccccgagatagccaagtttcgaataactcacatagacaagctagtagaagatccaacctctctgaacatcgctatgggaatgagtccagcgaacttgttaaagactgaggttaaaaaatgcttaatcgaatcaagacaaaccatcaggaaccaggtgattaaggatgcaaccatatatttgtatcatgaagaggatcggctcagaagtttcttatggtcaataaatcctctgttccctagatttttaagtgaattcaaatcaggcacttttttgggagtcgcagacgggctcatcagtctatttcaaaattctcgtactattcggaactcctttaagaaaaagtatcatagggaattggatgatttgattgtgaggagtgaggtatcctctttgacacatttagggaaacttcatttgagaaggggatcatgtaaaatgtggacatgttcagctactcatgctgacacattaagatacaaatcctggggccgtacagttattgggacaactgtaccccatccattagaaatgttgggtccacaacatcgaaaagagactccttgtgcaccatgtaacacatcagggttcaattatgtttctgtgcattgtccagacgggatccatgacgtctttagttcacggggaccattgcctgcttatctagggtctaaaacatctgaatctacatctattttgcagccttgggaaagggaaagcaaagtcccactgattaaaagagctacacgtcttagagatgctatctcttggtttgttgaacccgactctaaactagcaatgactatactttctaacatccactctttaacaggcgaagaatggaccaaaaggcagcatgggttcaaaagaacagggtctgcccttcataggttttcgacatctcggatgagccatggtgggttcgcatctcagagcactgcagcattgaccaggttgatggcaactacagacaccatgagggatctgggagatcagaatttcgactttttattccaagcaacgttgctctatgctcaaattaccaccactgttgcaagagacggatggatcaccagttgtacagatcattatcatattgcctgtaagtcctgtttgagacccatagaagagatcaccctggactcaagtatggactacacgcccccagatgtatcccatgtgctgaagacatggaggaatggggaaggttcgtggggacaagagataaaacagatctatcctttagaagggaattggaagaatttagcacctgctgagcaatcctatcaagtcggcagatgtataggttttctatatggagacttggcgtatagaaaatctactcatgccgaggacagttctctatttcctctatctatacaaggtcgtattagaggtcgaggtttcttaaaagggttgctagacggattaatgagagcaagttgctgccaagtaatacaccggagaagtctggctcatttgaagaggccggccaacgcagtgtacggaggtttgatttacttgattgataaattgagtgtatcacctccattcctttctcttactagatcaggacctattagagacgaattagaaacgattccccacaagatcccaacctcctatccgacaagcaaccgtgatatgggggtgattgtcagaaattacttcaaataccaatgccgtctaattgaaaagggaaaatacagatcacattattcacaattatggttattctcagatgtcttatccatagacttcattggaccattctctatttccaccaccctcttgcaaatcctatacaagccatttttatctgggaaagataagaatgagttgagagagctggcaaatctttcttcattgctaagatcaggagaggggtgggaagacatacatgtgaaattcttcaccaaggacatattattgtgtccagaggaaatcagacatgcttgcaagttcgggattgctaaggataataataaagacatgagctatcccccttggggaagggaatccagagggacaattacaacaatccctgtttattatacgaccaccccttacccaaagatgctagagatgcctccaagaatccaaaatcccctgctgtccggaatcaggttgggccaattaccaactggcgctcattataaaattcggagtatattacatggaatgggaatccattacagggacttcttgagttgtggagacggctccggagggatgactgctgcattactacgagaaaatgtgcatagcagaggaatattcaatagtctgttagaattatcagggtcagtcatgcgaggcgcctctcctgagccccccagtgccctagaaactttaggaggagataaatcgagatgtgtaaatggtgaaacatgttgggaatatccatctgacttatgtgacccaaggacttgggactatttcctccgactcaaagcaggcttggggcttcaaattgatttaattgtaatggatatggaagttcgggattcttctactagcctgaaaattgagacgaatgttagaaattatgtgcaccggattttggatgagcaaggagttttaatctacaagacttatggaacatatatttgtgagagcgaaaagaatgcagtaacaatccttggtcccatgttcaagacggtcgacttagttcaaacagaatttagtagttctcaaacgtctgaagtatatatggtatgtaaaggtttgaagaaattaatcgatgaacccaatcccgattggtcttccatcaatgaatcctggaaaaacctgtacgcattccagtcatcagaacaggaatttgccagagcaaagaaggttagtacatactttaccttgacaggtattccctcccaattcattcctgatccttttgtaaacattgagactatgctacaaatattcggagtacccacgggtgtgtctcatgcggctgccttaaaatcatctgatagacctgcagatttattgaccattagccttttttatatggcgattatatcgtattataacatcaatcatatcagagtaggaccgatacctccgaaccccccatcagatggaattgcacaaaatgtggggatcgctataactggtataagcttttggctgagtttgatggagaaagacattccactatatcaacagtgtttagcagttatccagcaatcattcccgattaggtgggaggctgtttcagtaaaaggaggatacaagcagaagtggagtactagaggtgatgggctcccaaaagatacccgaacttcagactccttggccccaatcgggaactggatcagatctctggaattggtccgaaaccaagttcgtctaaatccattcaatgagatcttgttcaatcagctatgtcgtacagtggataatcatttgaaatggtcaaatttgcgaagaaacacaggaatgattgaatggatcaatagacgaatttcaaaagaagaccggtctatactgatgttgaagagtgacctacacgaggaaaactcttggagagattaaaaaatcatgaggagactccaaactttaagtatgaaaaaaactttgatccttaagaccctcttgtggtttttattttttatctggttttgtggtcttcgt

SEQ ID NO: 18 - protein, 511 aa, Vesicular stomatitis Indiana virus
MKCFLYLAFLFIGVNCKFTIVFPHNQKGNWKNVPSNYHYCPSSSDLNWHNDLIGTGLQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDRELSSLGKEGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSGLHLSSKAQVFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 19 - DNA, 1992 bp, Newcastle disease virus
acgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgat

SEQ ID NO: 20 - DNA, 15186 bp, Newcastle disease virus
accaaacagagaatccgtaagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctagataccacaggcacaccgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacagggcaagaccaaggtgctgagcgcagcatgggagaagcatgggagcatccagccaccggccagtcaagacaacctcgatcgacaggacagatctgacaaacaaccatccacacccgagcaaacgaccccgcacgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagccaggaaagactgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatgggggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctagaagcggcaatcctctctcgcttcctcagccccactgaatgatcgcgtaaccgtaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgactcgtggactgatagtaaggaagactcagtattcatcaccacctatggattcatctttcaagttgggaatgaggaagccactgtcggcatgatcgatgataaacccaagcgcgagttactttccgctgcgatgctctgcctaggaagcgtcccaaataccggagaccttgttgagctggcaagggcctgtctcactatgatggtcacatgcaagaagagtgcaactaatactgagagaatggttttctcagtagtgcaggcaccccaagtgctgcaaagctgtagggttgtggcaaataaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagattctggatcccggttggcgccctccaggtgcaggatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcacggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatgttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattaggactaatatcttaactaggccgcctgggaatggagattgggccagtctgtgcaacgacccatactctttcaattttgagactgttgcaagcccaaacattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagagacctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 21 - DNA, 1782 bp, Newcastle disease virus
acgggtagaagactctggatcccggttggcgccctccaggtgcaggatgggctccagaccttttaccaagaacccagcacctatgatgctgactatccgggtcgcgctggtattgagttgcatctgtccggcaaactccattgatggcaggccttttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcccaactagcagtggcagttgggaagatgcagcagtttgttaatgaccaatttaataaaacagctcaggaattagactgcataaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagt

SEQ ID NO: 22 - protein, 553 aa, Newcastle disease virus
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 23 - DNA, 1662 bp, Artificial Sequence
  NDV F3aa-modified fusion protein with L289A (nucleotide sequence)
atgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactgcaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 24 - DNA, 1662 bp, Artificial Sequence
  NDV F3aa-modifed fusion protein (nucleotide sequence)
atgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggagggcggagacagaggcgctttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtga

SEQ ID NO: 25 - protein, 553 aa, Artificial Sequence
  NDV F3aa-modified fusion protein (amino acid sequence)
MGSRPFTKNPAPMMLTIRVALVLSCICPANSIDGRPFAAAGIVVTGDKAVNIYTSSQTGSIIVKLLPNLPKDKEACAKAPLDAYNRTLTTLLTPLGDSIRRIQESVTTSGGRRQRRFIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGIGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 26 - DNA, 510 bp, Homo sapiens
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg